(19) United States
(12) Patent Application Publication
(10) Pub. No.: US 2024/0390259 A1
(43) Pub. Date: Nov. 28, 2024

(54) MASCARA FORMULATION

(71) Applicant: MARY KAY INC., Addison, TX (US)

(72) Inventors: Wanli ZHAO, Addison, TX (US); Swara HARNE, Addison, TX (US)

(73) Assignee: MARY KAY INC., Addison, TX (US)

(21) Appl. No.: 18/674,250

(22) Filed: May 24, 2024

Related U.S. Application Data
(60) Provisional application No. 63/469,164, filed on May 26, 2023.

(51) Int. Cl. A61K 8/92; A61Q 1/10

(52) U.S. Cl. CPC A61K 8/922; A61K 8/927; A61Q 1/10; A61K 2800/43

(57) ABSTRACT

The present invention relates generally to methods and compositions useful for defining the appearance of eyelashes and increasing the length of eyelashes. The composition comprises 3 to 50% by weight of water based on a total weight of the composition; 5 to 20% by weight of a combination of waxes based on the total weight of the composition, wherein the combination of waxes comprises paraffin wax, polyethylene, and carnauba wax; 4 to 15% by weight of a combination of film forming agents based on the total weight of the composition, wherein the combination of film forming agents comprises vinylpyrrolidone (VP)/eicosene copolymer and triacontanyl polyvinylpyrrolidone (PVP); 5 to 15% iron oxides based on the total weight of the composition; silica; tocopheryl acetate; and panthenol.

### CROSS-REFERENCE TO RELATED APPLICATIONS

This application claims the benefit of priority to U.S. Provisional Patent Application Ser. No. 63/469,164, filed May 26, 2023, hereby incorporated by reference in its entirety.

### BACKGROUND OF THE INVENTION

### A. Field of the Invention

The present invention relates generally to the fields of cosmetics. More particularly, it concerns a mascara formulation that can be used to define the appearance of eyelashes and increase the length of eyelashes.

### B. Description of Related Art

There are a variety of commercially available mascaras. Some of the commercially available mascaras claim to thicken or enhance the volume of eyelashes, and these formulations tend to have a greasy or “heavy” tactile feel due to their reliance on large amounts of occlusive ingredients such as waxes (e.g., lanolin) to create the thickening effect. Further, such formulations tend to coalesce or clump together, which results in eyelashes that have an uneven and unsightly visual appearance thereby defeating the purpose of using the mascara product. Additionally, a mascara that is designed to thicken or enhance the volume of eyelashes may not necessarily be suitable to define the appearance of eyelashes or increase the length of eyelashes.

### SUMMARY OF THE INVENTION

The present invention overcomes deficiencies in the art by providing mascara compositions that can lengthen lashes and define lashes without clumping or flaking.

In one instance, there is disclosed a mascara composition that can define the appearance of eyelashes and increase the length of eyelashes. In one instance the mascara composition above includes any one of, any combination of, or all of water; a combination of waxes, including paraffin wax, polyethylene, and carnauba wax; film forming agents including vinylpyrrolidone (VP)/eicosene copolymer and triacontanyl polyvinylpyrrolidone (PVP); iron oxides; silica; tocopheryl acetate; and panthenol. The amounts of the ingredients within the composition can vary (e.g., amounts can be as low as 0.000001% to as high as 80% w/w or any range therein).

In some embodiments, the composition includes: 3 to 50% by weight of water based on a total weight of the composition; 5 to 20% by weight of a combination of waxes based on the total weight of the composition, wherein the combination of waxes comprises paraffin wax, polyethylene, and carnauba wax; 4 to 15% by weight of a combination of film forming agents based on the total weight of the composition, wherein the combination of film forming agents comprises vinylpyrrolidone (VP)/eicosene copolymer and triacontanyl polyvinylpyrrolidone (PVP); 5 to 15% iron oxides based on the total weight of the composition; silica; tocopheryl acetate; and panthenol. The composition may further comprise one or more ingredients described herein. For example, the composition may comprise one or more additional ingredients selected from one or more conditioning agents, moisturizing agents, pH adjusters, structuring agents, inorganic salts, and preservatives.

In some embodiments, the combination of waxes further comprises beeswax. In some embodiments, the combination of film forming agents further comprises acrylates/ethylhexyl acrylate copolymer. In some embodiments, the combination of film forming agents further comprises polyvinylpyrrolidone.

In some embodiments, the composition comprises: 45 to 50% by weight of water based on the total weight of the composition; 5 to 10% by weight of the combination of waxes based on the total weight of the composition; and 10 to 15% by weight of a combination of film forming agents.

In some aspects, the composition comprises 1 to 5% by weight of paraffin wax; 0.1 to 1% by weight of polyethylene; 1 to 2% by weight of carnauba wax; 1 to 5% by weight of VP/eicosene copolymer; and 1 to 5% by weight of triacontanyl PVP. In some embodiments, the combination of waxes comprises 1 to 5% by weight of beeswax. In some embodiments, the combination of film forming agents comprises 1 to 5% by weight of acrylates/ethylhexyl acrylate copolymer. In some aspects, the combination of film forming agents comprises 1 to 5% by weight of polyvinylpyrrolidone. In some embodiments, the composition comprises 0.02 to 2% by weight of silica; 0.01 to 1% by weight of tocopheryl acetate; and 0.01 to 1% by weight of panthenol.

In some aspects, the composition comprises any one of, any combination of, or all of glyceryl stearate; cellulose; behenyl alcohol; and propanediol. The amounts of the ingredients within the composition can vary (e.g., amounts can be as low as 0.000001% to as high as 80% w/w or any range therein). In some embodiments, the composition comprises 1 to 8% by weight of glyceryl stearate; 1 to 6% by weight of cellulose; 0.5 to 4% by weight of behenyl alcohol; and 0.5 to 3% by weight of propanediol. In some embodiments, the composition comprises any one of, any combination of, or all of stearic acid; hydrogenated cyclopentadiene; and trisiloxane. The amounts of the ingredients within the composition can vary (e.g., amounts can be as low as 0.000001% to as high as 80% w/w or any range therein). In some aspects, the composition comprises 1 to 8% by weight of stearic acid; 1 to 8% by weight of hydrogenated cyclopentadiene; and 1 to 8% by weight of trisiloxane.

In some embodiments, the composition comprises any one of, any combination of, or all of potassium hydroxide; phenoxyethanol; polyimide-1; disteardimonium hectorite; hydroxyacetophenone; caprylyl glycol; hydroxyethylcellulose; isopropyl titanium triisostearate; and xanthan gum. The amounts of the ingredients within the composition can vary (e.g., amounts can be as low as 0.000001% to as high as 80% w/w or any range therein). In some aspects, the composition comprises 0.01 to 1% by weight of potassium hydroxide; 0.01 to 1% by weight of phenoxyethanol; 0.01 to 1% by weight of polyimide-1; 0.01 to 1% by weight of disteardimonium hectorite; 0.01 to 1% by weight of caprylyl glycol; 0.01 to 1% by weight of hydroxyethylcellulose; 0.01 to 1% by weight of isopropyl titanium triisostearate; and 0.01 to 1% by weight of xanthan gum. In some aspects, the composition comprises any one of, any combination of, or all of laureth-21; triethyl citrate; disodium EDTA; benzyl alcohol; ethylhexylglycerin; hexylene glycol; aminomethyl propanol; tocopherol; and salicylic acid. The amounts of the ingredients within the composition can vary (e.g., amounts can be as low as 0.000001% to as high as 80% w/w or any range therein). In some aspects, the composition comprises 0.01 to 1% by weight of laureth-21; 0.01 to 1% by weight of triethyl citrate; 0.01 to 1% by weight of disodium EDTA; 0.01 to 1% by weight of benzyl alcohol; 0.01 to 1% by weight of ethylhexylglycerin; 0.01 to 1% by weight of hexylene glycol; 0.01 to 1% by weight of aminomethyl propanol; 0.01 to 1% by weight of tocopherol; and 0.01 to 1% by weight of salicylic acid.

In some embodiments, the composition further comprises isododecane; quaternium-90 bentonite; and propylene carbonate. In some embodiments, the composition comprises 25 to 75% by weight of isododecane; 1 to 10% by weight of quaternium-90 bentonite; and 0.5 to 5% by weight of propylene carbonate. In further embodiments, composition comprises 45 to 55% by weight of isododecane; 3.5 to 5.5% by weight of quaternium-90 bentonite; and 0.75 to 1.25% by weight of propylene carbonate. In some embodiments, the composition further comprises: 5 to 10% by weight of polymethylsilsesquioxane; 1 to 5% by weight of rayon; and 0.01 to 1% by weight of phenoxyethanol. In some aspects, the composition comprises any one of, any combination of, or all of hydrogenated cyclopentadiene; caprylyl glycol; and isopropyl titanium triisostearate. The amounts of the ingredients within the composition can vary (e.g., amounts can be as low as 0.000001% to as high as 80% w/w or any range therein). In some embodiments, the composition comprises 0.01 to 1% by weight of hydrogenated cyclopentadiene; 0.01 to 1% by weight of caprylyl glycol; and 0.01 to 1% by weight of isopropyl titanium triisostearate.

In one instance, the composition includes: 25 to 75% by weight of water based on the total weight of the emulsion; 5 to 20% by weight of a combination of waxes based on the total weight of the emulsion, wherein the combination of waxes includes paraffin wax, beeswax, and carnauba wax; film forming agents comprising 1 to 20% by weight of acrylates copolymer and/or acrylates/ethylhexyl acrylate copolymer, 1 to 5% by weight of VP/Eicosene copolymer and/or PVP/Eicosene copolymer, and 0.1 to 3% by weight of polyvinylpyrrolidone; 1 to 8% by weight of glyceryl stearate; 1 to 6% by weight of Acacia senegal gum extract; 1 to 6% by weight of cellulose; 0.5 to 4% by weight of behenyl alcohol; and 0.5 to 3% by weight of propanediol.

In one instance, the composition further includes any one of, any combination of, or all of: potassium hydroxide; phenoxyethanol, caprylyl glycol, ethylhexylglycerin, and/or hexylene glycol; hydroxyacetophenone; and xanthan gum. The amounts of the ingredients within the composition can vary (e.g., amounts can be as low as 0.000001% to as high as 80% w/w or any range therein). In one instance, the composition includes: 0.01 to 1.5% by weight of potassium hydroxide; 0.01 to 3% by weight of phenoxyethanol, caprylyl glycol, ethylhexylglycerin, and/or hexylene glycol; 0.01 to 1% by weight of hydroxyacetophenone; and 0.01 to 1% by weight of xanthan gum.

In one instance, the composition further includes any one of, any combination of, or all of iron oxides, stearic acid, and silica. The amounts of the ingredients within the composition can vary (e.g., amounts can be as low as 0.000001% to as high as 80% w/w or any range therein). In one instance, the composition includes: 3 to 20% by weight of iron oxides, 1 to 6% by weight of stearic acid, and 0.1 to 3% by weight of silica. The composition may further comprise one or more ingredients described herein. For example, the composition may comprise one or more additional ingredients selected from one or more conditioning agents, moisturizing agents, pH adjusters, structuring agents, inorganic salts, and preservatives.

In one instance, the composition further includes any one of, any combination of, or all of hydroxyethylcellulose, disodium EDTA, panthenol, and tocopheryl acetate. The amounts of the ingredients within the composition can vary (e.g., amounts can be as low as 0.000001% to as high as 80% w/w or any range therein). In one instance, the composition includes: 0.01 to 1% by weight of hydroxyethylcellulose, 0.01 to 1% by weight of disodium EDTA, 0.01 to 1% by weight of panthenol, and 0.01 to 1% by weight of tocopheryl acetate. The composition may further comprise one or more ingredients described herein. For example, the composition may comprise one or more additional ingredients selected from one or more conditioning agents, moisturizing agents, pH adjusters, structuring agents, inorganic salts, and preservatives.

In another aspect, the composition is adapted to define the appearance of a person's eyelash hair or fiber and/or to increase the length, bulk, and/or volume of a person's eyelash hair or fiber. The composition can also include pigments, dyes, colorants, etc., to achieve a desired color of the composition. The composition can be water resistant and/or smudge-resistant. The composition can be adapted to form a smooth surface when applied to a keratinous fiber. In some instances, the composition can be included on/loaded onto an applicator tip of a mascara wand and then applied to eyelashes.

Water-resistance (or being water-resistant) means that the composition has the ability to remain on a hair shaft despite being subjected to removal by water such as rinsing of a face, rain, or tears. Smudge-resistance (or being smudge-resistant) means that the composition has the ability to remain on a hair shaft despite being subjected to frictional forces such as normal rubbing of the eye area with one's hand.

The compositions disclosed herein can increase the length of a hair shaft, which can be measured by determining the length of a hair shaft prior to and post application of a given composition (e.g., a mascara composition). The compositions disclosed herein can also increase the volume of a hair shaft by 2 times, 3 times, 4 times, or 5 times the original volume of the hair shaft, with volume being measured by standard mathematical equations for the shape of the shaft (e.g., cylinder, irregular prism, rectangle, etc., as the shape of a given shaft may vary, so may the appropriate equation).

In some instances, the compositions disclosed herein (e.g., a mascara composition) may include a suitable pigment. Such pigments may include inorganic pigments, organic lake pigments, pearlescent pigments, and mixtures thereof. In some instances, the inorganic pigments may be selected from the group consisting of rutile and anatase titanium dioxide, black, yellow, red and brown iron oxides, manganese violet, ultramarine blue, chromium oxide, chromium hydrate, or ferric blue; or any combination thereof.

Methods of use for the compositions disclosed herein are also disclosed. In one aspect, the compositions disclosed herein are used in a method of defining the appearance of eyelashes and increasing the length of eyelashes. In some aspects, the method comprises applying a mascara composition as disclosed herein to the eyelashes, wherein application of the composition to the eyelashes defines the appearance of the eyelashes and increases the length of the eyelashes and wherein the composition comprises 3 to 50% by weight of water based on a total weight of the composition; 5 to 20% by weight of a combination of waxes based on the total weight of the composition, wherein the combination of waxes comprises paraffin wax, polyethylene, and carnauba wax; 4 to 15% by weight of a combination of film forming agents based on the total weight of the composition, wherein the combination of film forming agents comprises vinylpyrrolidone (VP)/eicosene copolymer and triacontanyl polyvinylpyrrolidone (PVP); 5 to 15% iron oxides based on the total weight of the composition; silica; tocopheryl acetate; and panthenol.

In some instances, the method comprises topically applying any one of the compositions disclosed herein to the eyelashes. In one aspect, any one of the compositions disclosed herein are applied and the composition is left on the application area, removed from the application area after a period of time, and/or removed directly after application. In some instances, the method may include using a mascara wand to apply the compositions disclosed herein to eyelash hairs or fibers.

An apparatus for applying the compositions disclosed herein is also disclosed. In one aspect, the apparatus includes a mascara wand and the compositions disclosed herein in contact with the mascara wand. In another aspect, the apparatus can be used in the methods disclosed herein. In some instances, the apparatus may include a base and a cap, with the cap further including an applicator such as a wand with a brush, comb, or wire structure at the distal end of the wand. In some instances, the wand may include an elongated portion having an applicator tip. The applicator tip can be designed to hold the compositions disclosed herein and to transfer the compositions from the tip to eyelashes. The applicator's tip can be straight or curved. The thickness or volume of the eyelash becomes greater after application of the compositions to the eyelash hair or fiber.

In particular aspects, the compositions of the present invention are formulated as a topical skin and/or hair composition (e.g., a composition for application to a person's eyelash hair). The composition can have a dermatologically acceptable vehicle or carrier for the compounds, compositions and extracts. The compositions can further include a moisturizing agent or a humectant, a surfactant, a silicone containing compounds, a UV agent, an oil, and/or other ingredients identified in this specification or those known in the art. The composition can be a lotion, cream, body butter, mask, scrub, wash, gel, serum, emulsion (e.g., oil-in-water, water-in-oil, wax-in-water, oil- and wax-in-water, silicone-in-water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, etc. The compositions can be in powdered form (e.g., dried, lyophilized, particulate, etc.). The compositions can be formulated for topical skin application at least 1, 2, 3, 4, 5, 6, 7, or more times a day during use. In some aspects of the present invention, compositions can be storage-stable or color-stable, or both. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

The compositions of the present invention can also be modified to have a desired oxygen radical absorbance capacity (ORAC) value. In certain non-limiting aspects, the compositions of the present invention or the component or extracts thereof identified throughout this specification can be modified to have an ORAC value per mg of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 95, 100, 200, 300, 400, 500, 600, 700, 800, 900, 1000, 2000, 3000, 4000, 5000, 6000, 7000, 8000, 9000, 10000, 15000, 20000, 30000, 50000, 100000 or more or any range derivable therein.

The compositions, in non-limiting aspects, can have a pH of about 6 to about 9. In some aspects, the pH can be 1, 2, 3, 4, 5, 6, 7, 8, 9, 10, 11, 12, 13, or 14. The compositions can include a triglyceride. Non-limiting examples include small, medium, and large chain triglycerides. In certain aspects, the triglyceride is a medium chain triglyceride (e.g., caprylic capric triglyceride). The compositions can also include preservatives. Non-limiting examples of preservatives include phenoxyethanol, methylparaben, propylparaben, or any mixture of thereof. In some embodiments, the composition is paraben-free.

Compositions of the present invention can have UVA and UVB absorption properties. The compositions can have an sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions can be sunscreen lotions, sprays, or creams.

The compositions of the present invention can also include any one of, any combination of, or all of the following additional ingredients: water, a conditioning agent, a chelating agent, a moisturizing agent, a pH adjuster, inorganic salts, a preservative, a thickening agent, a silicone containing compound, an essential oil, a structuring agent, a vitamin, a pharmaceutical ingredient, or an antioxidant, or any combination of such ingredients or mixtures of such ingredients. In certain aspects, the composition can include at least two, three, four, five, six, seven, eight, nine, ten, or more, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present invention are also contemplated. In certain embodiments, the composition is comprised in a container. The container can be a bottle, dispenser, or package. In some instances, the container can include a base and a cap, with the cap further including an applicator such as a wand with a brush, comb, or wire structure at the distal end of the wand. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

It is also contemplated that the compositions disclosed throughout this specification can be used as a leave-on or rinse-off composition. By way of example, a leave-on composition can be one that is applied to an eyelash and remains on the eyelash for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the eyelash and then removed or rinsed from the eyelash within a period of time such as less than 5, 4, 3, 2, or 1 minute. An example of a rinse off composition can be a skin cleanser, shampoo, conditioner, or soap. An example of a leave-on composition can be a mascara, skin moisturizer, sunscreen, mask, overnight cream, or a day cream.

It is contemplated that any embodiment discussed in this specification can be implemented with respect to any method or composition of the invention, and vice versa. Furthermore, compositions of the invention can be used to achieve methods of the invention.

In the context of the present invention, at least the following 71 Aspects are described. Aspect 1 includes a method of defining the appearance of eyelashes and increasing the length of eyelashes, the method comprising applying an oil- and wax-in-water emulsion to the eyelashes, wherein application of the emulsion to the eyelashes defines the appearance of the eyelashes and increases the length of the eyelashes and wherein the emulsion comprises: 25 to 75% by weight of water based on the total weight of the emulsion; 5 to 20% by weight of a combination of waxes based on the total weight of the emulsion, wherein the combination of waxes comprises paraffin wax, beeswax, and carnauba wax; film forming agents comprising acrylates copolymer and/or acrylates/ethylhexyl acrylate copolymer, VP/Eicosene copolymer and/or PVP/Eicosene copolymer, and polyvinylpyrrolidone; glyceryl stearate; Acacia senegal gum extract; cellulose; behenyl alcohol; and propanediol. Aspect 2 depends on Aspect 1, wherein the emulsion comprises: 1 to 20% by weight of acrylates copolymer and/or acrylates/ethylhexyl acrylate copolymer; 1 to 5% by weight of VP/Eicosene copolymer and/or PVP/Eicosene copolymer; and 0.1 to 3% by weight of polyvinylpyrrolidone. Aspect 3 depends on Aspect 1, wherein the emulsion comprises: 1 to 8% by weight of glyceryl stearate; 1 to 6% by weight of Acacia senegal gum extract; 1 to 6% by weight of cellulose; 0.5 to 4% by weight of behenyl alcohol; and 0.5 to 3% by weight of propanediol. Aspect 4 depends on Aspect 1, wherein the emulsion further comprises: potassium hydroxide; phenoxyethanol, caprylyl glycol, ethylhexylglycerin, and/or hexylene glycol; hydroxyacetophenone; and xanthan gum. Aspect 5 depends on Aspect 4, wherein the emulsion comprises: 0.01 to 1.5% by weight of potassium hydroxide; 0.01 to 3% by weight of phenoxyethanol, caprylyl glycol, ethylhexylglycerin, and/or hexylene glycol; 0.01 to 1% by weight of hydroxyacetophenone; and 0.01 to 1% by weight of xanthan gum. Aspect 6 depends on Aspect 1, wherein the emulsion further comprises: iron oxides; stearic acid; and silica. Aspect 7 depends on Aspect 6, wherein the emulsion comprises: 3 to 20% by weight of iron oxides; 1 to 6% by weight of stearic acid; and 0.1 to 3% by weight of silica. Aspect 8 depends on Aspect 1, wherein the emulsion further comprises: hydroxyethylcellulose; disodium EDTA; panthenol; and tocopheryl acetate. Aspect 9 depends on Aspect 8, wherein the emulsion comprises: 0.01 to 1% by weight of hydroxyethylcellulose; 0.01 to 1% by weight of disodium EDTA; 0.01 to 1% by weight of panthenol; and 0.01 to 1% by weight of tocopheryl acetate. Aspect depends on Aspect 1, wherein the emulsion is applied to the eyelashes with a mascara wand. Aspect 11 includes an oil- and wax-in-water emulsion to define the appearance of eyelashes and increase the length of eyelashes, the emulsion comprising: 25 to 75% by weight of water based on the total weight of the emulsion; 5 to 20% by weight of a combination of waxes based on the total weight of the emulsion, wherein the combination of waxes comprises paraffin wax, beeswax, and carnauba wax; film forming agents comprising acrylates copolymer and/or acrylates/ethylhexyl acrylate copolymer, VP/Eicosene copolymer and/or PVP/Eicosene copolymer, and polyvinylpyrrolidone; glyceryl stearate; Acacia senegal gum extract; cellulose; behenyl alcohol; and propanediol. Aspect 12 depends on Aspect 11, wherein the emulsion comprises: 1 to 20% by weight of acrylates copolymer and/or acrylates/ethylhexyl acrylate copolymer; 1 to 5% by weight of VP/Eicosene copolymer and/or PVP/Eicosene copolymer; and 0.1 to 3% by weight of polyvinylpyrrolidone. Aspect 13 depends on Aspect 12, wherein the emulsion comprises: 1 to 8% by weight of glyceryl stearate; 1 to 6% by weight of Acacia senegal gum extract; 1 to 6% by weight of cellulose; 0.5 to 4% by weight of behenyl alcohol; and 0.5 to 3% by weight of propanediol. Aspect 14 depends on Aspect 11, wherein the emulsion further comprises: potassium hydroxide; phenoxyethanol, caprylyl glycol, ethylhexylglycerin, and/or hexylene glycol; hydroxyacetophenone; and xanthan gum. Aspect depends on Aspect 14, wherein the emulsion comprises: 0.01 to 1.5% by weight of potassium hydroxide; 0.01 to 3% by weight of phenoxyethanol, caprylyl glycol, ethylhexylglycerin, and/or hexylene glycol; 0.01 to 1% by weight of hydroxyacetophenone; and 0.01 to 1% by weight of xanthan gum. Aspect 16 depends on Aspect 11, wherein the emulsion further comprises: iron oxides; stearic acid; and silica. Aspect 17 depends on Aspect 16, wherein the emulsion comprises: 3 to 20% by weight of iron oxides; 1 to 6% by weight of stearic acid; and 0.1 to 3% by weight of silica. Aspect 18 depends on Aspect 11, wherein the emulsion further comprises: hydroxyethylcellulose; disodium EDTA; panthenol; and tocopheryl acetate. Aspect 19 depends on Aspect 18, wherein the emulsion comprises: 0.01 to 1% by weight of hydroxyethylcellulose; 0.01 to 1% by weight of disodium EDTA; 0.01 to 1% by weight of panthenol; and 0.01 to 1% by weight of tocopheryl acetate. Aspect 20 is an apparatus for applying an emulsion on an eyelash, the apparatus comprising a mascara wand and the emulsion of Aspect 11 in contact with the mascara wand. Aspect 20 is a method of defining the appearance of eyelashes and increasing the length of eyelashes, the method comprising applying a mascara composition to the eyelashes, wherein application of the composition to the eyelashes defines the appearance of the eyelashes and increases the length of the eyelashes and wherein the composition comprises: 3 to 50% by weight of water based on a total weight of the composition; 5 to 20% by weight of a combination of waxes based on the total weight of the composition, wherein the combination of waxes comprises paraffin wax, polyethylene, and carnauba wax; 4 to 15% by weight of a combination of film forming agents based on the total weight of the composition, wherein the combination of film forming agents comprises vinylpyrrolidone (VP)/eicosene copolymer and triacontanyl polyvinylpyrrolidone (PVP); 5 to 15% iron oxides based on the total weight of the composition; silica; tocopheryl acetate; and panthenol. Aspect 21 depends on Aspect 20, wherein the combination of waxes further comprises beeswax. Aspect 22 depends on Aspect 1, wherein the combination of film forming agents further comprises acrylates/ethylhexyl acrylate copolymer. Aspect 23 depends on Aspect 20, wherein the combination of film forming agents further comprises polyvinylpyrrolidone. Aspect 24 depends on Aspect 20, wherein the composition comprises: 45 to 50% by weight of water based on the total weight of the composition; 5 to 10% by weight of the combination of waxes based on the total weight of the composition; and 10 to 15% by weight of a combination of film forming agents. Aspect 25 depends on Aspect 20, wherein the composition comprises: 1 to 5% by weight of paraffin wax; 0.1 to 1% by weight of polyethylene; 1 to 2% by weight of carnauba wax; 1 to 5% by weight of VP/eicosene copolymer; and 1 to 5% by weight of triacontanyl PVP. Aspect 26 depends on Aspect 21, wherein the combination of waxes comprises 1 to 5% by weight of beeswax. Aspect 27 depends on Aspect 22, wherein the combination of film forming agents comprises 1 to 5% by weight of acrylates/ethylhexyl acrylate copolymer. Aspect 28 depends on Aspect 23, wherein the combination of film forming agents comprises 1 to 5% by weight of polyvinylpyrrolidone. Aspect 29 depends on Aspect 20, wherein the composition comprises: 0.02 to 2% by weight of silica; 0.01 to 1% by weight of tocopheryl acetate; and 0.01 to 1% by weight of panthenol. Aspect 30 depends on Aspect 20, wherein the composition further comprises: glyceryl stearate; cellulose; behenyl alcohol; and propanediol. Aspect 31 depends on Aspect 20, wherein the composition comprises 1 to 8% by weight of glyceryl stearate; 1 to 6% by weight of cellulose; 0.5 to 4% by weight of behenyl alcohol; and 0.5 to 3% by weight of propanediol. Aspect 32 depends on Aspect 20, wherein the composition further comprises: stearic acid; hydrogenated cyclopentadiene; and trisiloxane. Aspect 33 depends on Aspect 32, wherein the composition comprises: 1 to 8% by weight of stearic acid; 1 to 8% by weight of hydrogenated cyclopentadiene; and 1 to 8% by weight of trisiloxane. Aspect 34 depends on Aspect 20, wherein the composition further comprises: potassium hydroxide; phenoxyethanol; polyimide-1; disteardimonium hectorite; hydroxyacetophenone; caprylyl glycol; hydroxyethylcellulose; isopropyl titanium triisostearate; and xanthan gum. Aspect 35 depends on Aspect 34, wherein the composition comprises: 0.01 to 1% by weight of potassium hydroxide; 0.01 to 1% by weight of phenoxyethanol; 0.01 to 1% by weight of polyimide-1; 0.01 to 1% by weight of disteardimonium hectorite; 0.01 to 1% by weight of caprylyl glycol; 0.01 to 1% by weight of hydroxyethylcellulose; 0.01 to 1% by weight of isopropyl titanium triisostearate; and 0.01 to 1% by weight of xanthan gum. Aspect 36 depends on Aspect 20, wherein the composition further comprises: laureth-21; triethyl citrate; disodium EDTA; benzyl alcohol; ethylhexylglycerin; hexylene glycol; aminomethyl propanol; tocopherol; and salicylic acid. Aspect 37 depends on Aspect 36, wherein the composition further comprises: 0.01 to 1% by weight of laureth-21; 0.01 to 1% by weight of triethyl citrate; 0.01 to 1% by weight of disodium EDTA; 0.01 to 1% by weight of benzyl alcohol; 0.01 to 1% by weight of ethylhexylglycerin; 0.01 to 1% by weight of hexylene glycol; 0.01 to 1% by weight of aminomethyl propanol; 0.01 to 1% by weight of tocopherol; and 0.01 to 1% by weight of salicylic acid. Aspect 38 depends on Aspect 20, wherein the composition further comprises: isododecane; quaternium-90 bentonite; and propylene carbonate. Aspect 39 depends on Aspect 38, wherein the composition comprises: 25 to 75% by weight of isododecane; 1 to 10% by weight of quaternium-90 bentonite; and 0.5 to 5% by weight of propylene carbonate. Aspect 40 depends on aspect 38, wherein the composition comprises: 45 to 55% by weight of isododecane; 3.5 to 5.5% by weight of quaternium-90 bentonite; and 0.75 to 1.25% by weight of propylene carbonate. Aspect 41 depends on Aspect 20, wherein the composition further comprises: polymethylsilsesquioxane; rayon; and phenoxyethanol. Aspect 42 depends on Aspect 41, wherein the composition further comprises: 5 to 10% by weight of polymethylsilsesquioxane; 1 to 5% by weight of rayon; and 0.01 to 1% by weight of phenoxyethanol. Aspect 43 depends on Aspect 20, wherein the composition further comprises: hydrogenated cyclopentadiene; caprylyl glycol; and isopropyl titanium triisostearate. Aspect 44 depends on Aspect 43, wherein the composition further comprises: 0.01 to 1% by weight of hydrogenated cyclopentadiene; 0.01 to 1% by weight of caprylyl glycol; and 0.01 to 1% by weight of isopropyl titanium triisostearate. Aspect 45 depends on Aspect 20, wherein the composition is applied to the eyelashes with a mascara wand. Aspect 46 is a mascara composition to define the appearance of eyelashes and increase the length of eyelashes, the composition comprising: 3 to 50% by weight of water based on a total weight of the composition; 5 to 20% by weight of a combination of waxes based on the total weight of the composition, wherein the combination of waxes comprises paraffin wax, polyethylene, and carnauba wax; 4 to 15% by weight of a combination of film forming agents based on the total weight of the composition, wherein the combination of film forming agents comprises vinylpyrrolidone (VP)/eicosene copolymer and triacontanyl polyvinylpyrrolidone (PVP); 5 to 15% iron oxides based on the total weight of the composition; silica; tocopheryl acetate; and panthenol. Aspect 47 is the composition of Aspect 46, wherein the combination of waxes further comprises beeswax. Aspect 48 is the composition of Aspect 46, wherein the combination of film forming agents further comprises acrylates/ethylhexyl acrylate copolymer. Aspect 49 is the composition of Aspect 46, wherein the combination of film forming agents further comprises polyvinylpyrrolidone. Aspect 50 is the composition of Aspect 46, wherein the composition comprises: 45 to 50% by weight of water based on the total weight of the composition; 5 to 10% by weight of the combination of waxes based on the total weight of the composition; and 10 to 15% by weight of a combination of film forming agents. Aspect 51 is the composition of Aspect 46, wherein the composition comprises: 1 to 5% by weight of paraffin wax; 0.1 to 1% by weight of polyethylene; 1 to 2% by weight of carnauba wax; 1 to 5% by weight of VP/eicosene copolymer; and 1 to 5% by weight of triacontanyl PVP. Aspect 52 is the composition of Aspect 47, wherein the combination of waxes comprises 1 to 5% by weight of beeswax. Aspect 53 is the composition of Aspect 48, wherein the combination of film forming agents comprises 1 to 5% by weight of acrylates/ethylhexyl acrylate copolymer. Aspect 54 is the composition of Aspect 49, wherein the combination of film forming agents comprises 1 to 5% by weight of polyvinylpyrrolidone. Aspect 55 is the composition of Aspect 46, wherein the composition comprises: 0.02 to 2% by weight of silica; 0.01 to 1% by weight of tocopheryl acetate; and 0.01 to 1% by weight of panthenol. Aspect 56 is the composition of Aspect 26, wherein the composition further comprises: glyceryl stearate; cellulose; behenyl alcohol; and propanediol. Aspect 57 is the composition of Aspect 46, wherein the composition comprises 1 to 8% by weight of glyceryl stearate; 1 to 6% by weight of cellulose; 0.5 to 4% by weight of behenyl alcohol; and 0.5 to 3% by weight of propanediol. Aspect 58 is the composition of any of Aspects 46 to 57, wherein the composition further comprises: stearic acid; hydrogenated cyclopentadiene; and trisiloxane. Aspect 59 is the composition of Aspect 58, wherein the composition comprises: 1 to 8% by weight of stearic acid; 1 to 8% by weight of hydrogenated cyclopentadiene; and 1 to 8% by weight of trisiloxane. Aspect 60 is the composition of any one of Aspects 46 to 59, wherein the composition further comprises: potassium hydroxide; phenoxyethanol; polyimide-1; disteardimonium hectorite; hydroxyacetophenone; caprylyl glycol; hydroxyethylcellulose; isopropyl titanium triisostearate; and xanthan gum. Aspect 61 is the composition of Aspect 60, wherein the composition comprises: 0.01 to 1% by weight of potassium hydroxide; 0.01 to 1% by weight of phenoxyethanol; 0.01 to 1% by weight of polyimide-1; 0.01 to 1% by weight of disteardimonium hectorite; 0.01 to 1% by weight of caprylyl glycol; 0.01 to 1% by weight of hydroxyethylcellulose; 0.01 to 1% by weight of isopropyl titanium triisostearate; and 0.01 to 1% by weight of xanthan gum. Aspect 62 is the composition of any of Aspects 46 to 61, wherein the composition further comprises: laureth-21; triethyl citrate; disodium EDTA; benzyl alcohol; ethylhexylglycerin; hexylene glycol; aminomethyl propanol; tocopherol; and salicylic acid. Aspect 63 is the composition of Aspect 62, wherein the composition comprises: 0.01 to 1% by weight of laureth-21; 0.01 to 1% by weight of triethyl citrate; 0.01 to 1% by weight of disodium EDTA; 0.01 to 1% by weight of benzyl alcohol; 0.01 to 1% by weight of ethylhexylglycerin; 0.01 to 1% by weight of hexylene glycol; 0.01 to 1% by weight of aminomethyl propanol; 0.01 to 1% by weight of tocopherol; and 0.01 to 1% by weight of salicylic acid. Aspect 64 is the composition of Aspect 46, wherein the composition further comprises: isododecane; quaternium-90 bentonite; and propylene carbonate. Aspect 65 is the composition of Aspect 64, wherein the composition comprises: 25 to 75% by weight of isododecane; 1 to 10% by weight of quaternium-90 bentonite; and 0.5 to 5% by weight of propylene carbonate. Aspect 66 is the composition of Aspect 64, wherein the composition comprises: 45 to 55% by weight of isododecane; 3.5 to 5.5% by weight of quaternium-90 bentonite; and 0.75 to 1.25% by weight of propylene carbonate. Aspect 67 is the composition of Aspect 46, wherein the composition further comprises: polymethylsilsesquioxane; rayon; and phenoxyethanol. Aspect 68 is the composition of Aspect 67, wherein the composition comprises: 5 to 10% by weight of polymethylsilsesquioxane; 1 to 5% by weight of rayon; and 0.01 to 1% by weight of phenoxyethanol. Aspect 69 is the composition of Aspect 46, wherein the composition further comprises: hydrogenated cyclopentadiene; caprylyl glycol; and isopropyl titanium triisostearate. Aspect 70 is the composition of Aspect 69, wherein the composition comprises: 0.01 to 1% by weight of hydrogenated cyclopentadiene; 0.01 to 1% by weight of caprylyl glycol; and 0.01 to 1% by weight of isopropyl titanium triisostearate. Aspect 71 is an apparatus for applying a mascara composition to an eyelash, the apparatus comprising a mascara wand and the mascara composition of any one of Aspects 46 to 70 in contact with the mascara wand.

In some embodiments, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

Also contemplated is a product comprising a composition of the present invention. In non-limiting aspects, the product can be a cosmetic product. The cosmetic product can be those described in other sections of this specification or those known to a person of skill in the art. Non-limiting examples of products include a mascara, a moisturizer, a cream, a lotion, a skin softener, a serum, a gel, a wash, a body butter, a scrub, a foundation, a night cream, a lipstick, a cleanser, a toner, a sunscreen, a mask, an anti-aging product, a deodorant, an antiperspirant, a perfume, a cologne, etc.

“Topical application” means to apply or spread a composition onto the surface of lips or keratinous tissue. “Topical skin composition” includes compositions suitable for topical application on skin, lips, and/or keratinous tissue. Such compositions are typically dermatologically-acceptable in that they do not have undue toxicity, incompatibility, instability, allergic response, and the like, when applied to lips, skin, and/or keratinous tissue. Topical skin care compositions of the present invention can have a selected viscosity to avoid significant dripping or pooling after application to lips, skin, and/or keratinous tissue.

“Keratinous tissue” includes keratin-containing layers disposed as the outermost protective covering of mammals and includes, but is not limited to, lips, skin, hair, and nails.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase, such as a measurable increase of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is the ability to define the appearance of eyelashes and increase the length of eyelashes.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: A drawing of a mascara container that can be used to apply the composition of the present invention to eyelashes.

### DESCRIPTION OF ILLUSTRATIVE EMBODIMENTS

As noted above, several of the unique aspects of the present invention provide the ability to define the appearance of eyelashes and increase the length of eyelashes without clumping and flaking.

The following subsections describe non-limiting aspects of the present invention in further detail.

The compositions disclosed herein are mascara compositions. The mascara compositions disclosed herein can be employed in methods of defining the appearance of eyelashes and increasing the length of eyelashes. The composition relies on a unique combination of any one of, any combination of, or all of water; a combination of waxes based on the total weight of the composition, wherein the combination of waxes comprises paraffin wax, polyethylene, and carnauba wax; a combination of film forming agents based on the total weight of the composition, wherein the combination of film forming agents comprises vinylpyrrolidone (VP)/eicosene copolymer and triacontanyl polyvinylpyrrolidone (PVP); iron oxides; silica; tocopheryl acetate; and panthenol. Non-limiting examples of such a composition are provided in Example 1 and Tables 1-4.

Some compositions disclosed herein can be applied to an eyelash and remain on the eyelash or hair for a period of time (e.g., at least 1, 2, 3, 4, 5, 10, 20, 30, or 60 minutes, or 6, 12, 18, 24, or 48 hours or more). After which, the composition, if needed, can be rinsed or cleaned from the eyelash or peeled from the eyelash.

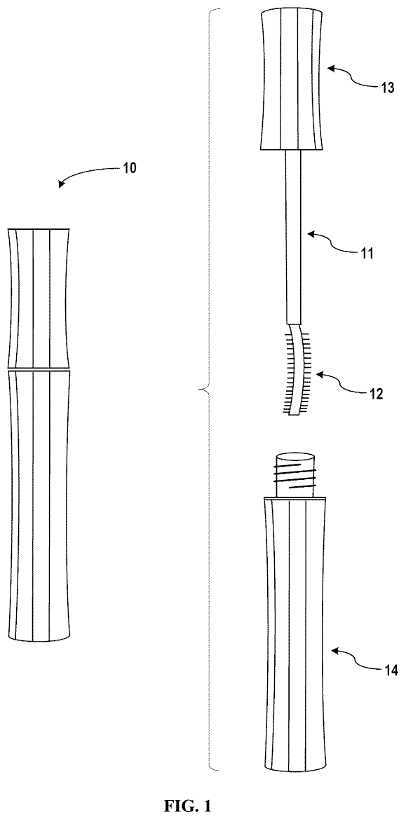

FIG. 1 illustrates a mascara container 10 that can be used to apply the compositions of the present invention to eyelashes. The container 10 may include an elongated portion or wand 11 and an applicator tip 12 coupled to the end of the wand 11 portion. The applicator tip 12 may be constructed into various shapes (e.g., straight, curved, or spiral), with various lengths and with various materials (e.g., plastic or silicone). The wand portion 11 can be coupled to a cap 13 of the container 10. The compositions of the present invention can be stored in the body or base 14 portion of the container 10.

These and other non-limiting aspects of the present invention are described in the following sections.

### A. KEY INGREDIENTS

The present invention is premised on a determination that a combination of one or more of key ingredients—panthenol, tocopheryl acetate, acrylates copolymer and/or acrylates/ethylhexyl acrylate copolymer, triacontanyl polyvinylpyrrolidone, paraffin, polyethylene, carnauba wax, beeswax, stearic acid, quaternium-90 bentonite, Tixogel-IDD, acrylates copolymer and/or acrylates/ethylhexyl acrylate copolymer, polyvinylpyrrolidone, glyceryl stearate, cellulose, hydroxyethyl cellulose, Acacia senegal gum extract, behenyl alcohol, propanediol, and xanthan gum—can be used to define the appearance of eyelashes and increase the length of eyelashes.

Panthenol, a provitamin of B5, is a conditioning agent. In some instances, this ingredient is commercially available. It has been determined that this ingredient can be used to attract moisture from the air and bind it to an eyelash hair and to protect, condition and strengthen eyelashes while preventing lash loss and breakage.

Tocopheryl acetate, a form of vitamin E, is an antioxidant. In some instances, this ingredient is commercially available, e.g., from Uniproma Chemical, under the trade name PromaCare® VEA. It has been determined that this ingredient can be used as an antioxidant for its conditioning benefits.

VP/Eicosene copolymer and/or PVP/Eicosene copolymer is a film former. In some instances, this ingredient is commercially available, e.g., from Givaudan, under the trade name Unimer U-15. It has been determined that this ingredient can be used to increase viscosity and impart structural characteristics to a cosmetic composition.

Triacontanyl polyvinylpyrrolidone (PVP) is a film former. In some instances, this ingredient is commercially available. It has been determined that this ingredient can be used to increase viscosity and impart structural characteristics to a cosmetic composition.

Paraffin is a solid mixture of hydrocarbons derived from petroleum, coal, or oil shale that consists of containing between 20 and 40 carbon atoms. It has been determined that this ingredient can be used to add structure and stability to a cosmetic. Paraffin can also be used to adjust the viscosity of a cosmetic composition.

Polyethylene is a polymer made of repeating ethylene units. It has been determined that this ingredient can be used to increase the viscosity, increase hardness, and raise the melting point of a cosmetics composition.

Carnauba wax is a natural, plant-based product that is made from the leaves of the Copernicia prunifera, a Brazilian palm tree. It has been determined that this ingredient can be used to increase the viscosity of cosmetic compositions, and to prevent liquid and oil elements from separating.

Beeswax is a wax that comes from the honeycombs of bees. It has been determined that this ingredient can be used as an emulsifier and emollient in cosmetic compositions.

Stearic acid is an 18 carbon fatty acid emulsifier. It has been determined that this ingredient can be used to aid in the blending of water and oil-based ingredients, prevent moisture loss, and soften skin.

Tixogel-IDD is a gel that includes propylene carbonate and quaternium-90 bentonite dispersed in isododecane. It has been determined that this ingredient can be used to impart thixotropic viscosity and helps suspend pigment particles in cosmetic compositions.

Quaternium-90 bentonite is a organoclay made of bentonite that has been modified to include the quaternary ammonium compound quaternium-90. It has been determined that this ingredient can be used to thicken cosmetic formulations, to reduce the tackiness of formulations, and to impart emolliency to cosmetic compositions.

Acrylates copolymer and/or acrylates/ethylhexyl acrylate copolymer is a film former. In some instances, this ingredient is commercially available. It has been determined that this ingredient can be used to confer water-resistant properties to a cosmetic composition.

Polyvinylpyrrolidone, also known as PVP, is a film former. In some instances, this ingredient is commercially available, e.g., from CISME, under the trade name COSMOROL™ K30. It has been determined that this ingredient can be used to stabilize an emulsion and to hold hair in shape.

Glyceryl stearate, also known as 2,3-dihydroxypropyl octadecenoate, is an emulsifying agent. In some instances, this ingredient is commercially available, e.g., from Evonik, under the trade name TEGIN® M Pellets or TEGIN® 4100 Pellets. It has been determined that this ingredient can be used as an emulsifier in cosmetic compositions.

Acacia senegal gum extract is a viscosity controlling agent. In some instances, this ingredient is commercially available, e.g., from Seppic, under the trade name SOLAGUM™ AX. It has been determined that this ingredient can be used to control viscosity of a cosmetic composition (e.g., as a thickening agent).

Cellulose is an absorbent. In some instances, this ingredient is commercially available, e.g., from Ashland, under the trade name Natrathix™ Bio Cellulose. It has been determined that this ingredient can be used to stabilize a cosmetic emulsion.

Hydroxyethyl cellulose is a modified fiber component of plants. It has been determined that this ingredient can be used as a thickening agent and to bind other ingredients within a cosmetic composition together.

Behenyl alcohol, also known as 1-docosanol, is an emulsion stabilizer. In some instances, this ingredient is commercially available, e.g., from BASF, under the trade name Lanette® 22. It has been determined that this ingredient can be used to stabilize a cosmetic emulsion.

Propanediol is a solvent. In some instances, this ingredient is commercially available, e.g., from Jover Scientech, under the trade name Vercatech PDIOL. It has been determined that this ingredient can be used as a solvent in cosmetic compositions.

Xanthan gum is a viscosity increasing agent. In some instances, this ingredient is commercially available, e.g., from CP Kelco, under the trade name KELTROL® Xanthan Gum. It has been determined that this ingredient can be used to control viscosity of a cosmetic composition (e.g., as a thickening agent).

This combination of ingredients can be used in different product forms to treat various skin conditions. By way of non-limiting examples, the combination of ingredients can be formulated in an emulsion (e.g., oil in water, water in oil), a gel, a serum, a gel emulsion, a gel serum, a lotion, a mask, a scrub, a wash, a cream, or a body butter.

The extracts described herein can be extracts made through extraction methods known in the art and combinations thereof. Non-limiting examples of extraction methods include the use of liquid-liquid extraction, solid phase extraction, aqueous extraction, ethyl acetate, alcohol, acetone, oil, supercritical carbon dioxide, heat, pressure, pressure drop extraction, ultrasonic extraction, etc. Extracts can be a liquid, solid, dried liquid, re-suspended solid, etc.

### B. AMOUNTS OF INGREDIENTS

It is contemplated that the compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### C. VEHICLES

The compositions of the present invention can include or be incorporated into all types of vehicles and carriers. The vehicle or carrier can be a pharmaceutically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

### D. STRUCTURE

The compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, masks, scrubs, body butters, peels, and ointments. Variations and other structures will be apparent to the skilled artisan and are appropriate for use in the present invention.

### E. ADDITIONAL INGREDIENTS

In addition to the combination of ingredients disclosed by the inventors, the compositions can also include additional ingredients such as cosmetic ingredients and pharmaceutical active ingredients. Non-limiting examples of these additional ingredients are described in the following subsections.

### 1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, iron oxides, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents (e.g., water, propanediol, and hexylene glycol), moisturizers (including, e.g., emollients, humectants, film formers, occlusive agents, and agents that affect the natural moisturization mechanisms of the skin), emulsion stabilizers (e.g., beeswax/cire d'abeille, behenyl alcohol, hydroxyethylcellulose), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatoires), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben, phenoxyethanol, and propylparaben), pH adjusters (e.g., sodium hydroxide, potassium hydroxide, and citric acid), absorbents (e.g., aluminum starch octenylsuccinate, kaolin, corn starch, oat starch, cellulose, cyclodextrin, silica, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, ethylhexylglycerin, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

a. UV Absorption and/or Reflecting Agents

UV absorption and/or reflecting agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate (octinoxate), isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidene camphor, and isopentyl 4-methoxycinnamate. Non-limiting examples of physical sunblocks include, kaolin, talc, petrolatum and metal oxides (e.g., titanium dioxide and zinc oxide).

b. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, hydroxyacetophenone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (see U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, laureth-21, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, and mixtures thereof.

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds include silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Michigan. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Michigan.

g. Exfoliating Agent

Exfoliating agents include ingredients that remove dead skin cells on the skin's outer surface. These agents may act through mechanical, chemical, and/or other means. Non-limiting examples of mechanical exfoliating agents include abrasives such as pumice, silica, cloth, paper, shells, beads, solid crystals, solid polymers, etc. Non-limiting examples of chemical exfoliating agents include acids and enzyme exfoliants. Acids that can be used as exfoliating agents include, but are not limited to, glycolic acid, lactic acid, citric acid, alpha hydroxy acids, beta hydroxy acids, etc. Other exfoliating agents known to those of skill in the art are also contemplated as being useful within the context of the present invention.

h. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang-ylang. Other essential oils known to those of skill in the art are also contemplated as being useful within the context of the present invention.

i. Thickening Agents

Thickening agents, including thickener or gelling agents, include substances which that can increase the viscosity of a composition. Thickeners include those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/VP copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerythritol (e.g., CARBOPOL™ 900 series from B. F. GOODRICH).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three units.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

j. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

k. Emollients

Useful emollients include the following: (a) silicone oils and modifications thereof such as linear and cyclic polydimethylsiloxanes; amino, alkyl, alkylaryl, and aryl silicone oils; (b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride; (c) waxes such as carnauba, spermaceti, beeswax, lanolin, and derivatives thereof; (d) hydrophobic plant extracts; (e) hydrocarbons such as liquid paraffins, vaseline, microcrystalline wax, ceresin, squalene, pristan and mineral oil; (f) higher fatty acids such as lauric, myristic, palmitic, stearic, behenic, oleic, linoleic, linolenic, lanolic, isostearic, arachidonic and poly unsaturated fatty acids (PUFA); (g) higher alcohols such as lauryl, cetyl, stearyl, oleyl, behenyl, cholesterol and 2-hexydecanol alcohol; (h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate; (i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils; (j) lipids such as cholesterol, ceramides, sucrose esters and pseudo-ceramides as described in European Patent Specification No. 556,957; (k) vitamins, minerals, and skin nutrients such as vitamins A, E, and K; vitamin alkyl esters, including vitamin C alkyl esters; magnesium, calcium, and milk; (l) sunscreens such as octyl methoxyl cinnamate (Parsol MCX) and butyl methoxy benzoylmethane (Parsol 1789); (l) phospholipids; (m) polyhydric alcohols such as glycerine and propylene glycol; and polyols such as caprylyl glycol and polyethylene glycols; (n) antiaging compounds such as alpha hydroxy acids, beta hydroxy acids; and (o) mixtures of any of the foregoing components, and the like.

l. Tackifiers

Examples of suitable tackifiers, include, but are not limited to, aliphatic hydrocarbon resins, aromatic modified aliphatic hydrocarbon resins, hydrogenated polycyclopentadiene resins, polycyclopentadiene resins, gum rosins, gum rosin esters, wood rosins, wood rosin esters, tall oil rosins, tall oil rosin esters, polyterpenes, aromatic modified polyterpenes, terpene phenolics, aromatic modified hydrogenated polycyclopentadiene resins, hydrogenated aliphatic resin, hydrogenated aliphatic aromatic resins, hydrogenated terpenes and modified terpenes, hydrogenated rosin acids, hydrogenated rosin esters, polyisoprene, partially or fully hydrogenated polyisoprene, polybutenediene, partially or fully hydrogenated polybutenediene, and the like. As is evidenced by some of the cited examples, the tackifier may be fully or partially hydrogenated. The tackifier may also be non-polar. (Non-polar meaning that the tackifier is substantially free of monomers having polar groups. Preferably, the polar groups are not present, however, if they are present, they are preferably present in an amount of up to about 5% by weight, preferably up to about 2% by weight, and more preferably up to about 0.5% by weight).

m. Colorant

The compositions of the present invention also contain at least one cosmetically acceptable colorant such as a pigment or dyestuff. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface-treated within the scope of the present invention but are not limited to treatments such as silicones, perfluorinated compounds, lecithin, and amino acids.

n. Surfactant

Surfactants useful as the surfactant components in the compositions of the present invention include nonionic, anionic, cationic, and amphoteric (zwitterionic) surfactants and may be used in combination with each other.

### o. pH Adjustors

The pH adjustors, include inorganic and organic acids and bases and in particular aqueous ammonia, citric acid, phosphoric acid, acetic acid, sodium hydroxide, lactic acid, levulinic acid, glycolic acid, tartaric acid, malic acid, pyrrolidonecarboxylic acid (PCA), succinic acid, citric acid, glutamic acid, 2-amino-2-methyl-1-propanol (AMP), and triethanolamine (TEA).

p. Reducing Agents

Suitable reducing agents include, but are not limited to, thiourea, salts (such as sodium salts) of thiosulfate, sulfite, bisulfite, metabisulfite, borohydride, and hypophosphite, ascorbic acid and salts, esters, and derivatives thereof (e.g., ascorbyl palmitate and ascorbyl polypeptide), and tocopherols and salts, esters, and derivatives thereof (e.g., tocopherol acetate). Other reducing agents are listed on pages 1655-56 of the INCI Handbook.

q. Fragrances

The compositions disclosed herein may optionally include a fragrance. Examples of possible fragrances include natural oils or naturally derived materials, and synthetic fragrances such as hydrocarbons, alcohols, aldehydes, ketones, esters, lactones, ethers, nitriles, and polyfunctionals. Non-limiting examples of natural oils include the following: basil (Ocimum basilicum) oil, bay (pimento acris) oil, bee balm (Monarda didyma) oil, bergamot (Citrus aurantium bergamia) oil, cardamom (Elettaria cardamomum) oil, cedarwood (Cedrus atlantica) oil, chamomile (Anthemis nobilis) oil, cinnamon (Cinnamomum cassia) oil, citronella (Cymbopogon nardus) oil, clary (Salvia sclarea) oil, clove (Eugenia caryophyllus) oil, cloveleaf (Eufenia caryophyllus) oil, Cyperus esculentus oil, cypress (Cupressus sempervirens) oil, Eucalyptus citriodora oil, geranium maculatum oil, ginger (Zingiber officinale) oil, grapefruit (Citrus grandis) oil, hazel (Corylus avellana) nut oil, jasmine (Jasminum officinale) oil, Juniperus communis oil, Juniperus oxycedrus tar, Juniperus virginiana oil, kiwi (Actinidia chinensis) water, lavandin (Lavandula hybrida) oil, lavender (Lavandula angustifolia) oil, lavender (Lavandula angustifolia) water, lemon (Citrus medica limonum) oil, lemongrass (Cymbopogon schoenanthus) oil, lime (Citrus aurantifolia) oil, linden (Tilia cordata) oil, linden (Tilia cordata) water, mandarin orange (Citrus nobilis) oil, nutmeg (Myristica fragrans) oil, orange (Citrus aurantium dulcis) flower oil, orange (Citrus aurantium dulcis) oil, orange (Citrus aurantium dulcis) water, patchouli (Pogostemon cablin) oil, peppermint (menthe piperita) oil, peppermint (Menthe peperita) water, rosemary (Rosmarinus officinalis) oil, rose oil, rose (Rosa damascena) extract, rose (Rosa multiflora) extract, rosewood (Aniba rosaeodora) extract, sage (Salvia officinalis) oil, sandalwood (Santalum album) oil, spearmint (Menthe viridis) oil, tea tree (Melaleuca alternifolia) oil, and ylang (Cananga odorata) oil. Some non-limiting examples of synthetic hydrocarbon fragrances include caryophyllene, β-farnesene, limonene, α-pinene, and, 3-pinene. Some non-limiting examples of synthetic alcohol fragrances include bacdanol, citronellol, linalool, phenethyl alcohol, and α-terpineol (R═H). Some non-limiting examples of synthetic aldehyde fragrances include 2-methyl undecanal, citral, hexyl cinnamic aldehyde, isocycolcitral, lilial, and 10-undecenal. Some non-limiting examples of synthetic ketone fragrances include cashmeran, α-ionone, isocyclemone E, koavone, muscone, and tonalide. Some non-limiting examples of synethetic ester fragrances include benzyl acetate, 4-t-butylcyclohexyl acetate (cis and trans), cedryl acetate, cyclacet, isobornyl acetate, and α-terpinyl acetate (R=acetyl). Some non-limiting examples of synthetic lactone fragrances include coumarin, jasmine lactone, muskalactone, and peach aldehyde. Some non-limiting examples of synthetic ether fragrances include ambroxan, anther, and galaxolide. Some non-limiting examples of synthetic nitrile fragrances include cinnamonitrile and gernonitrile. Finally, some non-limiting examples of synthetic polyfunctional fragrances include amyl salicylate, isoeugenol, hedione, heliotropine, lyral, and vanillin.

r. Foaming Agents

The foaming agents include, for example, sodium lauryl sulfate, sodium lauroyl sarcosine, sodium alkyl sulfosuccinates, sodium coconut oil fatty acid monoglycerol sulfonates, sodium α-olefin sulfonates, N-acylamino acid salts such as N-acyl glutamate, 2-alkyl-N-carboxymethyl-N-hydroxyethylimidazolinium betaine, maltitol fatty acid esters, sucrose fatty acid esters, polyglycerol fatty acid esters, fatty acid diethanolamides, polyoxyethylene sorbitan monostearate, polyoxyethylene hydrogenated castor oil and polyoxyethylene fatty acid esters. These foaming agents are usable either alone or in combination of two or more of them.

s. Tanning Agents

Suitable tanning agents include, without limitation, alpha-hydroxy aldehydes and ketones, glyceraldehyde and related alcohol aldehydes, various indoles, imidazoles and derivatives thereof, and various approved pigmentation agents. Other suitable tanning agents include, without limitation, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde.

t. Astringents

Suitable astringents include, without limitation, aluminum citrate, aluminum lactate, extracts of birch, extracts of coffee, extracts of evening primrose, extracts of grape, extracts of henna, extracts of ivy, extracts of lemon, extracts of witch hazel, Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof. What is meant by an extract is either the whole fruit, bean, and/or plant or select constituents of such fruit, bean, and/or plant.

u. Antiseptics

Suitable antiseptics include, without limitation, methyl, ethyl, propyl, or butyl ester of p-oxybenzoic acid, phenoxyethanol, o-phenylphenol, dehydroacetic acid, or salts thereof, p-cresol, m-cresol, o-chlor-m-xylenol, peppermint oil, Echinacea, bloodroot, cayenne, tea tree oil, wild bergamont, chaparral, stinging metal, bay, myrrh, rhatany bark, toothache tree, calendula, chamomile, mupirocin, neomycin sulfate, bacitracin, polymyxin B, I-ofloxacin, tetracyclines (chlortetracycline hydrochloride, oxytetracycline hydrochloride and tetrachcycline hydrochoride), clindamycin phsphate, gentamicin sulfate, benzalkonium chloride, benzethonium chloride, hexylresorcinol, methylbenzethonium chloride, phenol, quaternary ammonium compounds, triclocarbon, triclosan, and tea tree oil.

v. Deodorants and Antiperspirants

Suitable antiperspirants and deodorants include, without limitation, zinc salts such as zinc sulfate and zinc chloride, glycinates such as aluminum zirconium glycinate, aluminum chlorohydrate, aluminum zirconium tetrachlorohydrex, zinc carbonate, orthophenylphenol, and quaternary ammonium compounds such as dimethyl benzyl ammonium chloride and hexamethonium chloride.

w. Lighteners

Examples of skin lighteners include, without limitation, hydroquinone, kojic acid, licorice and/or its derivatives, ascorbic acid and/or its derivatives, arbutin, bearberry extract, Glycyrrhiza glabra and its derivatives, Chlorella vulgaris extract, perilla extract, coconut fruit extract, and/or other depigmenting agents.

x. Biocides

Examples of biocides include, without limitation, triclosan, 3,4,4′-trichlorocarbanilide (triclocarban); 3,4,4′-trifluoromethyl-4,4′-dichlorocarbanilide (cloflucarban); 5-chloro-2-methyl-4-isothiazolin-3-one; iodopropynlbutylcarbamate; 8-hydroxyquinoline; 8-hydroxyquinoline citrate; 8-hydroxyquinoline sulfate; 4-chloro-3,5-xylenol (chloroxylenol); 2-bromo-2-nitropropane-1,3-diol; diazolidinyl urea; butoconazole; nystatin; terconazole; nitrofurantoin; phenazopyridine; acyclovir; clortrimazole; chloroxylenol; chlorhexidine; miconazole; terconazole; butylparaben; ethylparaben; methylparaben; methylchloroisothiazoline; methylisothiazoline; a mixture of 1,3-bis(hydroxymethyl)-5,5-dimethylhydantoin and 3-iodo-2-propynyl butyl carbamate; oxyquinoline; EDTA; tetrasodium EDTA; p-hydroxyl benzoic acid ester; alkyl pyridinum compounds; coco phosphatidyl PG-dimonium chloride; chlorhexidine gluconate; chlorhexidine digluconate; chlorhexidine acetate; chlorhexidine isethionate; chlorhexidine hydrochloride; benzalkonium chloride; benzethonium chloride; polyhexamethylene biguanide; and mixtures thereof.

### 2. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### F. KITS

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the compositions, methods, and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions, methods, and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### 1. Example 1 (Exemplary Formulations)

Table 1 describes a non-limiting mascara formulation of the present invention. The Table 1 formulation is an oil- and wax-in-water emulsion that can define the appearance of eyelashes and increase the length of eyelashes by application of the emulsion to eyelash hairs.

TABLE 1*

| | % Concentration |
|---|---|
| Ingredient | (by weight) |
| Water | 50 |
| Iron oxides | 10 |
| Acrylates/ethylhexyl acrylate copolymer | 5 |
| Glyceryl stearate | 3 |
| Paraffin | 3 |
| Beeswax/cire d'abeille | 3 |
| Acacia senegal gum extract | 3 |
| Cellulose | 3 |
| Copernicia cerifera (carnauba) wax/cire de carnauba | 3 |
| Stearic acid | 3 |
| VP/Eicosene copolymer | 3 |
| Behenyl alcohol | 3 |
| Propanediol | 1 |
| Polyvinylpyrrolidone (PVP) | 1 |
| Silica | 1 |
| Potassium hydroxide | 0.5 |
| Phenoxyethanol | 10.5 |
| Hydroxyacetophenone | 0.5 |
| Caprylyl glycol | 10.1 |
| Hydroxyethylcellulose | 0.1 |
| Disodium EDTA | 0.1 |
| Panthenol | 0.1 |
| Tocopheryl acetate | 0.1 |
| Xanthan gum | 0.1 |
| Excipients** | q.s. |

*Formulation can be prepared by mixing the ingredients in a beaker under heat 70-75° C. until homogenous.
Subsequently, the formulation can be cooled to standing room temperature (20-25° C.).
Further, and if desired, additional ingredients can be added, for example, to modify the rheological properties of the composition.
**Excipients can be added, for example, to modify the rheological properties of the composition.
Alternatively, the amount of water can be varied so long as the amount of water in the composition is at least 20% w/w, and preferably between 30 to 70% w/w.

In one instance, the exemplary formulation in Table 1 was prepared by standard techniques known in the cosmetic industry. In particular, the water phase ingredients along chelating agents, preservatives, and gums are heated up to 80° C. Meanwhile, the oil phase ingredients are heated up to 80° C. in a separate beaker. When both phases are at the desired temperature, the oil phase is added to the water phase and homogenized for 5 minutes. Heat-sensitive ingredients are added in the cool down phase and the composition is cooled to 33° C. In one instance, excipients were added to the formulation to achieve stability (e.g., chelating agents and preservatives). In some instances, no excipients are added and the formulation can be q.s.'d with water.

Table 2 describes another non-limiting mascara formulation of the present invention. The Table 2 formulation is an oil- and wax-in-water emulsion that can define the appearance of eyelashes and increase the length of eyelashes by application of the emulsion to eyelash hairs.

TABLE 2*

| | % Concentration |
|---|---|
| Ingredient | (by weight) |
| Water | 50 |
| Iron oxides (black) | 10 |
| Acrylates copolymer | 10 |
| Glyceryl stearate | 4 |
| Paraffin | 4 |
| Beeswax/cire d'abeille | 3 |
| Acacia senegal gum extract | 3 |
| Cellulose | 3 |
| Copernicia cerifera (carnauba) wax/cire de carnauba | 3 |
| Stearic acid | 3 |
| PVP/Eicosene copolymer | 3 |
| Behenyl alcohol | 2 |
| Propanediol | 2 |
| Polyvinylpyrrolidone (PVP) | 1 |
| Nonporous silica | 1 |
| Potassium hydroxide (45% solution) | 1 |
| Phenoxyethanol, caprylyl glycol, ethylhexylglycerin, | 10.8 |
| hexylene glycol | |
| Hydroxyacetophenone | 0.3 |
| Hydroxyethylcellulose (250 HHR) | 0.2 |
| Disodium EDTA | 0.1 |
| Panthenol | 0.1 |
| Tocopheryl acetate | 0.1 |
| Xanthan gum | 0.1 |
| Excipients** | q.s. |

*Formulation can be prepared by mixing the ingredients in a beaker under heat 70-75° C. until homogenous.
Subsequently, the formulation can be cooled to standing room temperature (20-25° C.).
Further, and if desired, additional ingredients can be added, for example, to modify the rheological properties of the composition.
**Excipients can be added, for example, to modify the rheological properties of the composition.
Alternatively, the amount of water can be varied so long as the amount of water in the composition is at least 20% w/w, and preferably between 30 to 70% w/w.

In one instance, the exemplary formulation in Table 2 was prepared by standard techniques known in the cosmetic industry. In particular, the water phase ingredients along chelating agents, preservatives, and gums are heated up to 80° C. Meanwhile, the oil phase ingredients are heated up to 80° C. in a separate beaker. When both phases are at the desired temperature, the oil phase is added to the water phase and homogenized for 5 minutes. Heat-sensitive ingredients are added in the cool down phase and the composition is cooled to 33° C. In one instance, excipients were added to the formulation to achieve stability (e.g., chelating agents and preservatives). In some instances, no excipients are added and the formulation can be q.s.'d with water.

Table 3 describes another non-limiting mascara formulation of the present invention. The Table 3 formulation is an emulsion that can define the appearance of eyelashes and increase the length of eyelashes by application of the emulsion to eyelash hairs.

TABLE 3*

| | % Concentration |
|---|---|
| Ingredient | (by weight) |
| Water | 48.6 |
| Iron oxides (black) | 9.8 |
| Acrylates/ethylhexyl acrylate copolymer | 4.8 |
| Glyceryl stearate | 4 |
| Paraffin | 4 |
| VP/eicosene copolymer | 4 |
| Stearic acid | 3.5 |
| Beeswax/cire d'abeille | 3.2 |
| Hydrogenated polycyclopentadiene | 3.2 |
| Cellulose | 3 |
| Polyvinylpyrrolidone (PVP) | 2 |
| Triacontanyl PVP | 12 |
| Trisiloxane | 1.6 |
| Copernicia cerifera (carnauba) wax/cire de carnauba | 1.5 |
| Propanediol | 1 |
| Silica | 1 |
| Polyethylene | 0.5 |
| Potassium hydroxide | 10.5 |
| Phenoxyethanol | 10.4 |
| Polyimide-1 | 0.3 |
| Disteardimonium hectorite | 0.3 |
| Hydroxyacetophenone | 0.3 |
| Caprylyl glycol | 0.2 |
| Hydroxyethylcellulose | 0.2 |
| Isopropyl titanium triisostearate | 0.2 |
| Xanthan gum | 0.2 |
| Laureth-21 | 0.1 |
| Triethyl citrate | 0.1 |
| Disodium EDTA | 0.1 |
| Panthenol | 0.1 |
| Tocopheryl acetate | 0.1 |
| Benzyl alcohol | 0.1 |
| Ethylhexylglycerin | 0.1 |
| Hexylene glycol | 0.1 |
| Aminomethyl propanol | 0.1 |
| Excipients** | q.s. |

*Formulation can be prepared by mixing the ingredients in a beaker under heat 70-75° C. until homogenous.
Subsequently, the formulation can be cooled to standing room temperature (20-25° C.).
Further, and if desired, additional ingredients can be added, for example, to modify the rheological properties of the composition.
**Excipients can be added, for example, to modify the rheological properties of the composition.
Alternatively, the amount of water can be varied so long as the amount of water in the composition is at least 20% w/w, and preferably between 30 to 70% w/w.

In one instance, the exemplary formulation in Table 3 was prepared by standard techniques known in the cosmetic industry. In particular, the water phase ingredients along chelating agents, preservatives, and gums are heated up to 80° C. Meanwhile, the oil phase ingredients are heated up to 80° C. in a separate beaker. When both phases are at the desired temperature, the oil phase is added to the water phase and homogenized for 5 minutes. Heat-sensitive ingredients are added in the cool down phase and the composition is cooled to 33° C. In one instance, excipients were added to the formulation to achieve stability (e.g., chelating agents and preservatives). In some instances, no excipients are added and the formulation can be q.s.'d with water.

Table 4 describes another non-limiting mascara formulation of the present invention. The Table 4 formulation is a water-proof mascara composition that can define the appearance of eyelashes and increase the length of eyelashes.

TABLE 4*

| | % Concentration |
|---|---|
| Ingredient | (by weight) |
| Isododecane | 50.2 |
| Iron oxides (black) | 8.8 |
| Polymethylsilsesquioxane | 7.2 |
| Copernicia cerifera (carnauba) wax/cire de carnauba | 5 |
| Paraffin | 5 |
| Quaternium-90 bentonite | 4 |
| Polyethylene | 4 |
| Water/eau | 4 |
| VP/eicosane copolymer | 3 |
| Rayon | 2 |
| Silica | 1.5 |
| Triacontanyl PVP | 1.5 |
| Propylene carbonate | 1 |
| Phenoxyethanol | 0.8 |
| Hydrogenated cyclopentadiene | 0.7 |
| Caprylyl glycol | 0.7 |
| Isopropyl titanium triisostearate | 0.2 |
| Tocopheryl acetate | 0.1 |
| Panthenol | 0.05 |
| Excipients** | q.s. |

*Formulation can be prepared by mixing the ingredients in a beaker under heat 70-75° C. until homogenous.
Subsequently, the formulation can be cooled to standing room temperature (20-25° C.).
Further, and if desired, additional ingredients can be added, for example, to modify the rheological properties of the composition.
**Excipients can be added, for example, to modify the rheological properties of the composition.
Alternatively, the amount of water can be varied so long as the amount of water in the composition is at least 20% w/w, and preferably between 30 to 70% w/w.

In one instance, the exemplary formulation in Table 4 was prepared by standard techniques known in the cosmetic industry. In particular, the oil phase ingredients including waxes and film formers are combined in a vessel, mixed, and heated up to a 100° C. After the oil phase is completely melted, hydrogenated cyclopentadiene is added followed by isododecane. Iron oxides is added at 100° C. and the vessel contents are homogenized to ensure uniform dispersion. Then filler, preservative, and rheology modifier are added to the vessel followed by a second homogenization step. The vessel contents are maintained at 75° C., then rayon and silica are added. The vessel contents are then cooled to room temperature and aerated as the final step.

### 2. Example 2 (Assays)

Assays that can be used to determine the efficacy of any one of the ingredients or any combination of ingredients or compositions having said combination of ingredients disclosed throughout the specification and claims can be determined by methods known to those of ordinary skill in the art. The following are non-limiting assays that can be used in the context of the present invention. It should be recognized that other testing procedures can be used, including, for example, objective and subjective procedures.

B16 Pigmentation Assay: Melanogenesis is the process by which melanocytes produce melanin, a naturally produced pigment that imparts color to skin, hair, and eyes. Inhibiting melanogenesis is beneficial to prevent skin darkening and lighten dark spots associated with aging. This bioassay utilizes B16-F1 melanocytes (ATCC), an immortalized mouse melanoma cell line, to analyze the effect of compounds on melanogenesis. The endpoint of this assay is a spectrophotometric measurement of melanin production and cellular viability. B16-F1 melanocytes, can be cultivated in standard DMEM growth medium with 10% fetal bovine serum (MEDIATECH) at 37° C. in 10% CO₂ and then treated with any one of the active ingredients, combination of ingredients, or compositions having said combinations disclosed in the specification for 6 days. Following incubation, melanin secretion is measured by absorbance at 405 nm and cellular viability is quantified.

Collagen Stimulation Assay: Collagen is an extracellular matrix protein critical for skin structure. Increased synthesis of collagen helps improve skin firmness and elasticity. This bioassay can be used to examine the effect of any one of the active ingredients, combination of ingredients, or compositions having said combinations disclosed in the specification on the production of procollagen peptide (a precursor to collagen) by human epidermal fibroblasts. The endpoint of this assay is a spectrophotometric measurement that reflects the presence of procollagen peptide and cellular viability. The assay employs the quantitative sandwich enzyme immunoassay technique whereby a monoclonal antibody specific for procollagen peptide has been pre-coated onto a microplate. Standards and samples can be pipetted into the wells and any procollagen peptide present is bound by the immobilized antibody. After washing away any unbound substances, an enzyme-linked polyclonal antibody specific for procollagen peptide can be added to the wells. Following a wash to remove any unbound antibody-enzyme reagent, a substrate solution can be added to the wells and color develops in proportion to the amount of procollagen peptide bound in the initial step using a microplate reader for detection at 450 nm. The color development can be stopped and the intensity of the color can be measured.

For generation of samples and controls, subconfluent normal human adult epidermal fibroblasts (Cascade Biologics) cultivated in standard DMEM growth medium with 10% fetal bovine serum (MEDIATECH) at 37° C. in 10% CO₂, can be treated with each of the combination of ingredients or compositions having said combinations disclosed in the specification for 3 days. Following incubation, cell culture medium can be collected and the amount of procollagen peptide secretion quantified using a sandwich enzyme linked immuno-sorbant assay (ELISA) from TAKARA (#MK101).

Elastin Stimulation Assay: Elastin is a connective tissue protein that helps skin resume shape after stretching or contracting. Elastin is also an important load-bearing protein used in places where mechanical energy is required to be stored. Elastin is made by linking many soluble tropoelastin protein molecules, in a reaction catalyzed by lysyl oxidase. Elastin secretion and elastin fibers can be monitored in cultured human fibroblasts by staining of cultured human fibroblasts using immunofluorescent antibodies directed against elastin.

Laminin and Fibronectin Stimulation Assay: Laminin and fibronectin are major proteins in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin and fibronectin are two structural glycoproteins located in the DEJ. Considered the glue that holds the cells together, laminin and fibronectin are secreted by dermal fibroblasts to help facilitate intra- and inter-cellular adhesion of the epidermal calls to the DEJ. Laminin and fibronectin secretion can be monitored by quantifying laminin and fibronectin in cell supernatants of cultured human fibroblasts treated for 3 days with culture medium with or without 1.0% final concentration of the test ingredient(s). Following incubation, laminin and fibronectin content can be measured using immunofluorescent antibodies directed against laminin and antibodies directed against fibronectin in an enzyme linked immuno-sorbant assay (ELISA). Measurements are normalized for cellular metabolic activity, as determined by bioconversion of 3-(4,5-dimethylthiazol-2-yl)-5-(3-carboxymethoxyphenyl)-2-(4-sulfophenyl)-2H-tetrazolium (MTS).

Tumor Necrosis Factor Alpha (TNF-α) Assay: The prototype ligand of the TNF superfamily, TNF-α, is a pleiotropic cytokine that plays a central role in inflammation. Increase in its expression is associated with an up regulation in pro-inflammatory activity. This bioassay can be used to analyze the effect of any one of the active ingredients, combination of ingredients, or compositions having said combinations disclosed in the specification on the production of TNF-α by human epidermal keratinocytes. The endpoint of this assay can be a spectrophotometric measurement that reflects the presence of TNF-α and cellular viability. The assay employs the quantitative sandwich enzyme immunoassay technique whereby a monoclonal antibody specific for TNF-α has been pre-coated onto a microplate. Standards and samples can be pipetted into the wells and any TNF-α present is bound by the immobilized antibody. After washing away any unbound substances, an enzyme-linked polyclonal antibody specific for TNF-α can be added to the wells. Following a wash to remove any unbound antibody-enzyme reagent, a substrate solution can be added to the wells and color develops in proportion to the amount of TNF-α bound in the initial step using a microplate reader for detection at 450 nm. The color development can be stopped and the intensity of the color can be measured. Subconfluent normal human adult keratinocytes (Cascade Biologics) cultivated in EPILIFE™ standard growth medium (Cascade Biologics) at 37° C. in 5% CO₂, can be treated with phorbol 12-myristate 13-acetate (PMA, 10 ng/ml, SIGMA CHEMICAL, #P1585-1 MG) and any one of the active ingredients, combination of ingredients, or compositions having said combinations disclosed in the specification for 6 hours. PMA has been shown to cause a dramatic increase in TNF-α secretion which peaks at 6 hours after treatment. Following incubation, cell culture medium can be collected and the amount of TNF-α secretion quantified using a sandwich enzyme linked immuno-sorbant assay (ELISA) from R&D Systems (#DTA00C).

Antioxidant (AO) Assay: An in vitro bioassay that measures the total anti-oxidant capacity of any one of the ingredients, combination of ingredients, or compositions having said combinations disclosed in the specification. The assay relies on the ability of antioxidants in the sample to inhibit the oxidation of ABTS® (2,2′-azino-di-[3-ethylbenzthiazoline sulphonate]) to ABTS®.+ by metmyoglobin. The antioxidant system of living organisms includes enzymes such as superoxide dismutase, catalase, and glutathione peroxidase; macromolecules such as albumin, ceruloplasmin, and ferritin; and an array of small molecules, including ascorbic acid, α-tocopherol, β-carotene, reduced glutathione, uric acid, and bilirubin. The sum of endogenous and food-derived antioxidants represents the total antioxidant activity of the extracellular fluid. Cooperation of all the different antioxidants provides greater protection against attack by reactive oxygen or nitrogen radicals, than any single compound alone. Thus, the overall antioxidant capacity may give more relevant biological information compared to that obtained by the measurement of individual components, as it considers the cumulative effect of all antioxidants present in plasma and body fluids. The capacity of the antioxidants in the sample to prevent ABTS® oxidation is compared with that of Trolox, a water-soluble tocopherol analogue, and is quantified as molar Trolox equivalents. Anti-Oxidant capacity kit #709001 from CAYMAN CHEMICAL (Ann Arbor, Michigan USA) can be used as an in vitro bioassay to measure the total anti-oxidant capacity of each of any one of the active ingredients, combination of ingredients, or compositions having said combinations disclosed in the specification. The protocol can be followed according to manufacturer recommendations.

ORAC Assay: Oxygen Radical Absorption (or Absorbance) Capacity (ORAC) of any one of the active ingredients, combination of ingredients, or compositions having said combinations disclosed in the specification can also be assayed by measuring the antioxidant activity of such ingredients or compositions. Antioxidant activity indicates a capability to reduce oxidizing agents (oxidants). This assay quantifies the degree and length of time it takes to inhibit the action of an oxidizing agent, such as oxygen radicals, that are known to cause damage to cells (e.g., skin cells). The ORAC value of any one of the active ingredients, combination of ingredients, or compositions having said combinations disclosed in the specification can be determined by methods known to those of ordinary skill in the art (see U.S. Publication Nos. 2004/0109905 and 2005/0163880; and commercially available kits such as Zen-Bio ORAC Anti-oxidant Assay kit (#AOX-2)). The Zen-Bio ORAC Anti-oxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

Mushroom tyrosinase activity assay: In mammalian cells, tyrosinase catalyzes two steps in the multi-step biosynthesis of melanin pigments from tyrosine (and from the polymerization of dopachrome). Tyrosinase is localized in melanocytes and produces melanin (aromatic quinone compounds) that imparts color to skin, hair, and eyes. Purified mushroom tyrosinase (from SIGMA) can be incubated with its substrate L-Dopa (from FISHER) in the presence or absence of each of the active ingredients, any one of the combination of ingredients, or compositions having said combinations disclosed in the specification. Pigment formation can be evaluated by colorimetric plate reading at 490 nm. The percent inhibition of mushroom tyrosinase activity can be calculated compared to non-treated controls to determine the ability of test ingredients or combinations thereof to inhibit the activity of purified enzyme. Test extract inhibition was compared with that of kojic acid (SIGMA).

Matrix Metalloproteinase 3 and 9 Enzyme Activity (MMP3; MMP9) Assay: An in vitro matrix metalloprotease (MMP) inhibition assay. MMPs are extracellular proteases that play a role in many normal and disease states by virtue of their broad substrate specificity. MMP3 substrates include collagens, fibronectins, and laminin; while MMP9 substrates include collagen VII, fibronectins and laminin. Using Colorimetric Drug Discovery kits from BioMol International for MMP3 (AK-400) and MMP-9 (AK-410), this assay is designed to measure protease activity of MMPs using a thiopeptide as a chromogenic substrate (Ac-PLG-[2-mercapto-4-methyl-pentanoyl]-LG-OC2H5) 5,6. The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, which can be detected by its absorbance at 412 nm (8=13,600 M-1 cm-1 at pH 6.0 and above 7). The active ingredients, any one of the combination of ingredients, or compositions having said combinations disclosed in the specification can be assayed.

Matrix Metalloproteinase 1 Enzyme Activity (MMP1) Assay: An in vitro matrix metalloprotease (MMP) inhibition assay. MMPs are extracellular proteases that play a role in many normal and disease states by virtue of their broad substrate specificity. MMP1 substrates include collagen IV. The MOLECULAR PROBES ENZ/CHEK GELATINASE/COLLAGENASE ASSAY kit (#E12055) utilizes a fluorogenic gelatin substrate to detect MMP1 protease activity. Upon proteolytic cleavage, bright green fluorescence is revealed and may be monitored using a fluorescent microplate reader to measure enzymatic activity.

The ENZ/CHEK GELATINASE/COLLAGENASE ASSAY kit (#E12055) from Invitrogen is designed as an in vitro assay to measure MMP1 enzymatic activity. The active ingredients, any one of the combination of ingredients, or compositions having said combinations disclosed in the specification can be assayed. The assay relies upon the ability of purified MMP1 enzyme to degrade a fluorogenic gelatin substrate. Once the substrate is specifically cleaved by MMP 1 bright green fluorescence is revealed and may be monitored using a fluorescent microplate reader. Test materials are incubated in the presence or absence of the purified enzyme and substrate to determine their protease inhibitor capacity.

Cyclooxygenase (COX) Assay: An in vitro cyclooxygenase-1 and -2 (COX-1, -2) inhibition assay. COX is a bifunctional enzyme exhibiting both cyclooxygenase and peroxidase activities. The cyclooxygenase activity converts arachidonic acid to a hydroperoxy endoperoxide (Prostaglandin G2; PGG2) and the peroxidase component reduces the endoperoxide (Prostaglandin H2; PGH2) to the corresponding alcohol, the precursor of prostaglandins, thromboxanes, and prostacyclins. This COX Inhibitor screening assay measures the peroxidase component of cyclooxygenases. The peroxidase activity is assayed colorimetrically by monitoring the appearance of oxidized N,N,N′,N′-tetramethyl-p-phenylenediamine (TMPD). This inhibitor screening assay includes both COX-1 and COX-2 enzymes in order to screen isozyme-specific inhibitors. The Colormetric COX (ovine) Inhibitor screening assay (#760111, CAYMAN CHEMICAL) can be used to analyze the effects of each of the active ingredients, any one of the combination of ingredients, or compositions having said combinations disclosed in the specification on the activity of purified cyclooxygnase enzyme (COX-1 or COX-2). According to manufacturer instructions, purified enzyme, heme and test extracts can be mixed in assay buffer and incubated with shaking for 15 min at room temperature. Following incubation, arachidonic acid and colorimetric substrate can be added to initiate the reaction. Color progression can be evaluated by colorimetric plate reading at 590 nm. The percent inhibition of COX-1 or COX-2 activity can be calculated compared to non-treated controls to determine the ability of test extracts to inhibit the activity of purified enzyme.

Lipoxygenase (LO) Assay: An in vitro lipoxygenase (LO) inhibition assay. LOs are non-heme iron-containing dioxygenases that catalyze the addition of molecular oxygen to fatty acids. Linoleate and arachidonate are the main substrates for LOs in plants and animals. Arachadonic acid may then be converted to hydroxyeicosotrienenoic (HETE) acid derivatives, that are subsequently converted to leukotrienes, potent inflammatory mediators. This assay provides an accurate and convenient method for screening lipoxygenase inhibitors by measuring the hydroperoxides generated from the incubation of a lipoxygenase (5-, 12-, or 15-LO) with arachidonic acid. The Colorimetric LO Inhibitor screening kit (#760700, CAYMAN CHEMICAL) can be used to determine the ability of each of the active ingredients, any one of the combination of ingredients, or compositions having said combinations disclosed in the specification to inhibit enzyme activity. Purified 15-lipoxygenase and test ingredients can be mixed in assay buffer and incubated with shaking for 10 min at room temperature. Following incubation, arachidonic acid can be added to initiate the reaction and the mixtures can be incubated for an additional 10 min at room temperature. Colorimetric substrate can be added to terminate catalysis and color progression can be evaluated by fluorescence plate reading at 490 nm. The percent inhibition of lipoxygenase activity can be calculated compared to non-treated controls to determine the ability of each of the active ingredients, any one of the combination of ingredients, or compositions having said combinations disclosed in the specification to inhibit the activity of purified enzyme.

Elastase Assay: ENZCHEK® Elastase Assay (Kit #E-12056) from MOLECULAR PROBES (Eugene, Oregon USA) can be used as an in vitro enzyme inhibition assay for measuring inhibition of elastase activity for each of the active ingredients, any one of the combination of ingredients, or compositions having said combinations disclosed in the specification. The ENZCHEK kit contains soluble bovine neck ligament elastin that can be labeled with dye such that the conjugate's fluorescence can be quenched. The non-fluorescent substrate can be digested by elastase or other proteases to yield highly fluorescent fragments. The resulting increase in fluorescence can be monitored with a fluorescence microplate reader. Digestion products from the elastin substrate have absorption maxima at ˜505 nm and fluorescence emission maxima at ˜515 nm. The peptide, N-methoxysuccinyl-Ala-Ala-Pro-Val-chloromethyl ketone, can be used as a selective, collective inhibitor of elastase when utilizing the ENZCHEK ELASTASE ASSAY KIT for screening for elastase inhibitors.

Production of Ceramides: Ceramides in cell or tissue samples can be labeled with a mouse monoclonal antibody anti-ceramide (ENZO LIFE SCIENCE, ref ALX-804-196 clone MID15B4) diluted to 1/50 for 2 hours at room temperature with an amplifier system biotin/streptavidin. Video microscope observation can be performed to view ceramides (pink stain).

Oil Control Assay: An assay to measure reduction of sebum secretion from sebaceous glands and/or reduction of sebum production from sebaceous glands can be assayed by using standard techniques known to those having ordinary skill in the art. In one instance, the forehead can be used. Each of the active ingredients, any one of the combination of ingredients, or compositions having said combinations disclosed in the specification can be applied to one portion of the forehead once or twice daily for a set period of days (e.g., 1, 2, 3, 4, 5, 6, 7, 8, 9, 10, 11, 12, 13, 14, or more days), while another portion of the forehead is not treated with the composition. After the set period of days expires, then sebum secretion can be assayed by application of fine blotting paper to the treated and untreated forehead skin. This is done by first removing any sebum from the treated and untreated areas with moist and dry cloths. Blotting paper can then be applied to the treated and untreated areas of the forehead, and an elastic band can be placed around the forehead to gently press the blotting paper onto the skin. After 2 hours the blotting papers can be removed, allowed to dry and then transilluminated. Darker blotting paper correlates with more sebum secretion (or lighter blotting paper correlates with reduced sebum secretion).

Erythema Assay: An assay to measure the reduction of skin redness can be evaluated using a MINOLTA chroma meter. Skin erythema may be induced by applying a 0.2% solution of sodium dodecyl sulfate on the forearm of a subject. The area is protected by an occlusive patch for 24 hrs. After 24 hrs, the patch is removed and the irritation-induced redness can be assessed using the a* values of the MINOLTA chroma meter. The a* value measures changes in skin color in the red region. Immediately after reading, the area is treated with the active ingredients, any one of the combination of ingredients, or compositions having said combinations disclosed in the specification. Repeat measurements can be taken at regular intervals to determine the formula's ability to reduce redness and irritation.

Skin Moisture/Hydration Assay: Skin moisture/hydration benefits can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72° C.). Three separate impedance readings can be taken on each side of the face, recorded, and averaged. The T5 setting can be used on the impedance meter which averages the impedance values of every five seconds application to the face. Changes can be reported with statistical variance and significance. Each of the active ingredients, any one of the combination of ingredients, or compositions having said combinations disclosed in the specification can be assayed according to this process.

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether each of the active ingredients, any one of the combination of ingredients, or compositions having said combinations disclosed in the specification is inducing irritation. The measurements can be made on each side of the face and averaged, as left and right facial values. Skin clarity can also be measured using the Minolta Meter. The measurement is a combination of the a*, b, and L values of the Minolta Meter and is related to skin brightness, and correlates well with skin smoothness and hydration. Skin reading is taken as above. In one non-limiting aspect, skin clarity can be described as L/C where C is chroma and is defined as (a²+b²)^{1/2}.

Skin Dryness, Surface Fine Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Clinical Grading of Skin Tone Assay: Clinical grading of skin tone can be performed via a ten point analog numerical scale: (10) even skin of uniform, pinkish brown color. No dark, erythremic, or scaly patches upon examination with a hand held magnifying lens. Microtexture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaliness. Skin rough to the touch in some areas; and (1) uneven skin coloration and texture. Numerous areas of scaliness and discoloration, either hypopigmented, erythremic or dark spots. Large areas of uneven color more than 1 cm in diameter. Evaluations were made independently by two clinicians and averaged.

Clinical Grading of Skin Smoothness Assay: Clinical grading of skin smoothness can be analyzed via a ten point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations were made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman et al. (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Skin Firmness Assay with a Hargens Ballistometer: Skin firmness can be measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and are of the replicas covered by wrinkles or fine lines was determined.

Surface Contour of the Skin Assay with a Profilometer/Stylus Method: The surface contour of the skin can be measured by using the Profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Rₐ=Standardize roughness; lₘ=the traverse (scan) length; and y=the absolute value of the location of the profile relative to the mean profile height (x-axis).

MELANODERM™ Assay: In other non-limiting aspects, the efficacy of each of the active ingredients, any one of the combination of ingredients, or compositions having said combinations disclosed in the specification can be evaluated by using a skin analog, such as, for example, MELANODERM™. Melanocytes, one of the cells in the skin analog, stain positively when exposed to L-dihydroxyphenyl alanine (L-DOPA), a precursor of melanin. The skin analog, MELANODERM™, can be treated with a variety of bases containing each of the active ingredients, any one of the combination of ingredients, or compositions having said combinations disclosed in the specification or with the base alone as a control. Alternatively, an untreated sample of the skin analog can be used as a control.

Production of Filaggrin: Changes in the production of filaggrin in keratinocytes due to each of the active ingredients, any one of the combination of ingredients, or compositions having said combinations disclosed in the specification can be measured. Filaggrin is the precursor to Natural Moisturizing Factor (NMF) in the skin. Increased NMF increases the moisture content of the skin. Filaggrin production in treated and non-treated keratinocytes can be determined using a bioassay that analyzes filaggrin concentration in keratinocyte cell lysates. A non-limiting example of a bioassay that can be used to quantify filaggrin production is the PROTEINSIMPLE® SIMON™ western blotting protocol. For each sample, normal human epidermal keratinocytes (NHEK) are grown in EPI-200-MATTEK EPILIFE® growth media with calcium from Life Technologies (M-EP-500-CA). NHEK are incubated in growth medium overnight at 37° C. in 5% CO₂ prior to treatment. NHEK are then incubated in growth medium with 1% test compound/extract or no compound/extract (negative control) for 24 to 36 hours. The NHEK can then be washed, collected, and stored on ice or colder until lysed on ice using a lysis buffer and sonication. The protein concentrations of the samples can be determined and used to normalize the samples. The lysates can be stored at −80° C. until use in the quantification assay.

The PROTEINSIMPLE® SIMON™ western blotting bioassay assay employs a quantitative western blotting immunoassay technique using an antibody specific for filaggrin to quantitatively detect filaggrin in the test samples. Cell samples are lysed and normalized for protein concentration. Normalized samples and molecular weight standards can then be loaded and ran on a denatured protein separation gel using capillary electrophoresis. The proteins in the gel are immobilized and immunoprobed using a primary antibody specific for filaggrin. The immobilized proteins can then be immunoprobed with an enzyme-linked detection antibody that binds the primary antibody. A chemiluminescent substrate solution can then be added to the immobilized proteins to allow chemiluminescent development in proportion to the amount of filaggrin bound in the immobilization. The chemiluminescent development is stopped at a specific time and the intensity of the chemiluminescent signal can be measured and compared to positive and negative controls.

Production of Occludin: Changes in the production of occludin in keratinocytes due to each of the active ingredients, any one of the combination of ingredients, or compositions having said combinations disclosed in the specification can be measured. Occludin is a protein critical to the formulation of tight junctions and the skin's moisture barrier function. A non-limiting example of how occludin production in treated and non-treated keratinocytes can be determined is by the use of a bioassay that analyzes occludin concentration in keratinocyte cell lysates. The bioassay can be performed using PROTEINSIMPLE® SIMON™ western blotting protocol. For the samples, adult human epidermal keratinocytes (HEKa) from Life Technologies (C-005-5C) can be grown at 37° C. and 5% CO₂ for 24 hours in EPILIFE™ growth media with calcium from Life Technologies (M-EP-500-CA) supplemented with Keratinocyte Growth Supplement (HKGS) from Life Technologies (S-101-5). HEKa are then incubated in growth medium with test compound/extract, no compound/extract for negative control, or with 1 mM CaCl₂) for positive control for 24 to 48 hours. The HEKa are then washed, collected, and stored on ice or colder until lysed on ice using a lysis buffer and sonication. The protein concentrations of the samples can be determined and used to normalize the samples. The lysates are stored at −80° C. until use in the bioassay.

The PROTEINSIMPLE® SIMON™ western blotting bioassay assay employs a quantitative western blotting immunoassay technique using an antibody specific for occludin to quantitatively detect occludin in the test samples. Cell samples are lysed and normalized for protein concentration. Normalized samples and molecular weight standards are then loaded and ran on a denatured protein separation gel using capillary electrophoresis. The proteins in the gel are then immobilized and immunoprobed using a primary antibody specific for occludin. The immobilized proteins are immunoprobed with an enzyme-linked detection antibody that binds the primary antibody. A chemiluminescent substrate solution is then added to the immobilized proteins to allow chemiluminescent development in proportion to the amount of occludin bound in the immobilization. The chemiluminescent development can be stopped at a specific time and the intensity of the chemiluminescent signal can be measured and compared to positive and negative controls.

Keratinocyte Monolayer Permeability: Changes in the permeability of a keratinocyte monolayer due to each of the active ingredients, any one of the combination of ingredients, or compositions having said combinations disclosed in the specification can be measured. Keratinocyte monolayer permeability is a measure of skin barrier integrity. Keratinocyte monolayer permeability in treated and non-treated keratinocytes can be determined using, as a non-limiting example, the In Vitro Vascular Permeability assay by MILLIPORE (ECM642). This assay analyzes endothelial cell adsorption, transport, and permeability. Briefly, adult human epidermal keratinocytes from Life Technologies (C-005-5C) can be seeded onto a porous collagen-coated membrane within a collection well. The keratinocytes are then incubated for 24 hours at 37° C. and 5% CO₂ in EPILIFE™ growth media with calcium from LIFE TECHNOLOGIES (M-EP-500-CA) supplemented with Keratinocyte Growth Supplement (HKGS) from LIFE TECHNOLOGIES (S-101-5). This incubation time allows the cells to form a monolayer and occlude the membrane pores. The media is then replaced with fresh media with (test sample) or without (non-treated control) test compounds/extracts and the keratinocytes are incubated for an additional 48 hours at 37° C. and 5% CO₂. To determine permeability of the keratinocyte monolayer after incubation with/without the test compound/extract, the media is replaced with fresh media containing a high molecular weight Fluorescein isothiocyanate (FITC)-Dextran and the keratinocytes are incubated for 4 hours at 37° C. and 5% CO₂. During the 4 hours incubation, FITC can pass through the keratinocytes monolayer and porous membrane into the collection well at a rate proportional to the monolayer's permeability. After the 4 hour incubation, cell viability and the content of FITC in the collection wells can be determined. For the FITC content, the media in the collection well is collected and fluorescence of the media determined at 480 nm (Em) when excited at 520 nm. Percent permeability and percent change in comparison to the non-treated controls can be determined by the following equations: Percent Permeability=((Mean Ex/Em of test sample)/Mean Ex/Em untreated control)*100; Percent Change=Percent Permeability of test sample-Percent Permeability of untreated control.

Production of Hyaluronic Acid: Changes in the production of hyaluronic acid in human dermal fibroblasts due to each of the active ingredients, any one of the combination of ingredients, or compositions having said combinations disclosed in the specification can be measured. HA is a polysaccharide involved in stabilization of the structure of the matrix and is involved in providing turgor pressure to tissue and cells.

As one non-limiting example, HA production in treated and non-treated adult human dermal fibroblasts (HDFa) cells can be determined using the Hyaluronan DuoSet ELISA kit from R&D Systems (DY3614). In this assay, for production of samples, subconfluent HDFa cells from Cascade Biologics (C-13-5C) are incubated at 37° C. and 10% CO₂ in starvation medium (0.15% fetal bovine serum and 1% Penicillin Streptomycin solution in Dulbecco's Modified Eagle Medium) for 72 hours prior to treatment. The cells are then incubated with fresh starvation medium with either test compound, positive control (phorbol 12-myristate 13-acetate from SIGMA-ALDRICH (P1585) and platelet derived growth factor from SIGMA-ALDRICH (P3201)), or no additive for 24 hours. Media is then collected and frozen at −80° C. until use in the ELISA assay.

Briefly, the ELISA assay employs a quantitative sandwich enzyme immunoassay technique whereby a capture antibody specific for HA can be pre-coated onto a microplate. Standards and media from treated and untreated cells are pipetted into the microplate wells to enable any HA present to be bound by the immobilized antibody. After washing away any unbound substances, an enzyme-linked detection antibody specific for HA is added to the wells. Following a wash to remove any unbound antibody-enzyme reagent, a substrate solution is added to the wells to allow color development in proportion to the amount of HA bound in the initial step. The color development is stopped at a specific time and the intensity of the color at 450 nm can be measured using a microplate reader.

As another non-limiting example, human skin explants can be cultured in survival explants medium at 37° C. in a humidified atmosphere supplemented with 5% CO₂. Treatment of the explants can be carried out by topical application of sample product (n=3) on days D0, D2, D3, D6, D8, and D9. The control explants (n=3) receive no treatment except renewal of survival explants medium. Half of the volume of the survival medium can be renewed at days D3, D6, and D8. At D9, three explants of each condition can be taken and cut in half. A half explant is fixed in buffered formalin and the other is frozen at −80° C.

After 48 hours of fixation in ordinary Bouin and 24 hours in formalin, the samples can be dried and soaked in paraffin using an automatic tissue processor Leica TP 1020. Sections of 5 microns can be performed with a microtome (Minot type LEICA RM2125) and mounted on SUPERFROST™ histological slides. Microscopic observations can be performed by optical microscopy, using a LEICA ORTHOPLAN or LEICA DM LB microscope. Images can be taken with an OLYMPUS DP72 camera and CELL\hat{ }D software. General morphology can be examined on paraffin sections stained with Masson's trichrome Goldner variant. The staining of hyaluronic acid can be performed with an anti-hyaluronic acid biotinylated protein (HABP) (SEIKAGAKU ref 400763-1A) diluted to 1/100 for 1 hour at room temperature, with an amplifier system biotin/streptavidin (VECTOR, VECTASTAIN PK-7200).

Inhibition of Hyaluronidase Activity: Changes in the activity of hyaluronidase due to each of the active ingredients, any one of the combination of ingredients, or compositions having said combinations disclosed in the specification can be measured. Hyaluronidase is an enzyme that degrades HA. HA is a polysaccharide involved in stabilization of the structure of the matrix and is involved in providing turgor pressure to tissue and cells. As one non-limiting example, hyaluronidase activity can be determined using an in vitro protocol modified from SIGMA-ALDRICH protocol #EC 3.2.1.35. Briefly, hyaluronidase type 1-S from SIGMA-ALDRICH (H3506) is added to microplate reaction wells containing test compound or controls. Tannic acid can be used as a positive control inhibitor, no test compound can be added for the control enzyme, and wells with test compound or positive control but without hyaluronidase can be used as a background negative control. The wells are incubated at 37° C. for 10 minutes before addition of substrate (HA). Substrate is added and the reactions incubated at 37° C. for 45 minutes. A portion of each reaction solution is then transferred to and gently mixed in a solution of sodium acetate and acetic acid pH 3.75 to stop that portion of the reaction (stopped wells). The stopped wells and the reaction wells should both contain the same volume of solution after addition of the portion of the reaction solution to the stopped wells. Both the reaction wells and the stopped wells are incubated for 10 minutes at room temperature. Absorbance at 600 nm is then measured for both the reaction wells and the stopped wells. Inhibition can be calculated using the following formulas: Inhibitor (or control) activity=(Inhibitor stopped wells absorbance at 600 nm-inhibitor reaction wells absorbance at 600 nm); Initial activity=control enzyme absorbance at 600 nm; Percent Inhibition=[(Initial activity/Inhibitor Activity)*100]−100.

Peroxisome Proliferator-Activated Receptor Gamma (PPAR-γ) Activity: Changes in the activity of PPAR-γ due to each of the active ingredients, any one of the combination of ingredients, or compositions having said combinations disclosed in the specification can be measured. PPAR-γ is a receptor critical for the production of sebum. As one non-limiting example, the activity of PPAR-γ can be determined using a bioassay that analyzes the ability of a test compound or composition to inhibit binding of a ligand. Briefly, fluorescent small-molecule pan-PPAR ligand, FLUORMONE™ Pan-PPAR Green, available from Life Technologies (PV4894), can be used to determine if test compounds or compositions are able to inhibit binding of the ligand to PPAR-γ. The samples wells include PPAR-γ and fluorescent ligand and either: test compound or composition (test); a reference inhibitor, rosiglitazone (positive control); or no test compound (negative control). The wells are incubated for a set period of time to allow the ligand opportunity to bind the PPAR-γ. The fluorescence polarization of each sample well can then be measured and compared to the negative control well to determine the percentage of inhibition by the test compound or composition.

Cytokine array: Human epidermal keratinocytes are cultured to 70-80% confluency. The media in the plate is aspirated and 0.025% trypsin/EDTA is added. When the cells became rounded, the culture dish is gently tapped to release the cells. The trypsin/EDTA containing cells are removed from the culture dish and neutralized. Cells are centrifuged for 5 min. at 180×g to form a pellet of cells. The supernatant is aspirated. The resulting pellet is resuspended in EPILIFE™ media (Cascade Biologics). The cells are seeded in 6-well plates at approximately 10-20% confluency. After the cells became approximately 80% confluent, the media is aspirated and 1.0 ml of EPILIFE™, along with phorbol 13-Myristate 12-acetate (“PMA”) (a known inducer of inflammation) and the test composition dilutions are added to two replicate wells (i.e., 1.0% (100 μl of 100× stock) and 0.1% (10 μl of 100× stock) test compositions are diluted into a final volume of 1 ml EPILIFE™ Growth Medium). The media is gently swirled to ensure adequate mixing. In addition, 1.0 ml of EPILIFE™ is added to the control wells, with and without additional PMA. The plates are then incubated at 37±1° C. and 5.0+1% CO₂ for approximately 5 hours after dosing. Following this 5-hour incubation, all media is collected in conical tubes and frozen at −70° C.

For analysis, a 16-pad hybridization chamber is attached to 16-pad FAST slides arrayed in triplicate with 16 anti-cytokine antibodies plus experimental controls (WHATMAN BIOSCIENCES), and the slides are placed into a FASTFrame (4 slides per frame) for processing. Arrays are blocked for 15 min. at room temperature using 70 ml S&S PROTEIN ARRAY BLOCKING BUFFER (WHATMAN SCHLEICHER AND SCHEULL). Blocking buffer is removed and 70 ml of each supernatant sample is added to each array. Arrays are incubated for 3 hours at room temperature with gentle agitation. Arrays are washed 3 times with TBS-T. Arrays are treated with 70 ml of an antibody cocktail, containing one biotinylated antibody corresponding to each of the arrayed capture antibodies. Arrays are incubated for 1 hour at room temperature with gentle agitation. Arrays are washed 3 times with TBS-T. Arrays are incubated with 70 ml of a solution containing streptavidin-Cy5 conjugate for 1 hour at room temperature with gentle agitation. Arrays are washed 3 times with TBS-T, quickly rinsed in de-ionized water, and dried.

Slides can be imaged in a PERKIN-ELMER SCANARRAY 4000 confocal fluorescent imaging system. Array images can be saved and analyzed using IMAGING RESEARCH ARRAYVISION software. Briefly, spot intensities are determined by subtracting background signal. Spot replicates from each sample condition can be averaged and then compared to the appropriate controls.

Endothelial Tube Formation: Endothelial tube formation is involved in angiogenesis and micro-vessel capillary formation. Capillary formation and angiogenesis may contribute to redness and rosacea of the skin. The ability for endothelial cells to form tubes in the presence or absence of test extracts and compounds may be determined using a capillary tubule disruption assay with pre-formed primary human umbilical vein endothelial cells (HUVEC) in a cell culture system.

Briefly, HUVECs are cultured in vitro on Extracellular Matrix, which stimulates the attachment and tubular morphogenesis of endothelial cells to form capillary-like lumen structures. These in vitro formed capillary tubules are similar to human blood vessel capillaries in many aspects. The capillary tube assay is based on this phenomenon and is used for evaluation of potential vasculature targeting agents.

HUVEC cultures are grown in a 5% CO₂ 37° C. cell incubator. The full growth medium for HUVECs is Endothelial Cell Basal Medium (EBM) supplemented with 2% fetal bovine serum (FBS), 12 μg/ml bovine brain extract, 1 μg/ml hydrocortisone, and 1 μg/ml GA-1000 (gentamicin-amphothericin). HUVEC cultures between passage 3 and 8 may be used for all assay experiments.

HUVECs are pre-labeled with fluorescent agent Calcein AM and seeded in Extracellular Matrix coated 96-well culture plate with their full growth medium. After about four hours of the morphogenesis process, the endothelial capillary tubes should be formed. Then, test agent in designed doses in 50 μl volume is applied into the formed capillary tubule cultures as treatment conditions. The no-treatment controls can be added with vehicle of test agents. SUTENT®, a FDA approved anti-angiogenic drug one concentration can be included as assay performance control. After about six hours of treatment, the endothelial tubule morphology in each well is examined by microscopy, imaged, and the capillary disrupting activities under treatment conditions can be quantitatively analyzed. Each test conditions can be conducted in duplicate wells, including controls.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A mascara composition to define the appearance of eyelashes and increase the length of eyelashes, the composition comprising:
3 to 50% by weight of water based on a total weight of the composition;
5 to 20% by weight of a combination of waxes based on the total weight of the composition, wherein the combination of waxes comprises paraffin wax, polyethylene, and carnauba wax;
4 to 15% by weight of a combination of film forming agents based on the total weight of the composition, wherein the combination of film forming agents comprises vinylpyrrolidone (VP)/eicosene copolymer and triacontanyl polyvinylpyrrolidone (PVP);
5 to 15% iron oxides based on the total weight of the composition;
silica;
tocopheryl acetate; and
panthenol.

2. The composition of claim 1, wherein the combination of waxes further comprises beeswax.

3. The composition of claim 1, wherein the combination of film forming agents further comprises acrylates/ethylhexyl acrylate copolymer.

4. The composition of claim 1, wherein the combination of film forming agents further comprises polyvinylpyrrolidone.

5. The composition of claim 1, wherein the composition comprises:
45 to 50% by weight of water based on the total weight of the composition;
5 to 10% by weight of the combination of waxes based on the total weight of the composition; and
10 to 15% by weight of a combination of film forming agents.

6. The composition of claim 1, wherein the composition comprises:
1 to 5% by weight of paraffin wax;
0.1 to 1% by weight of polyethylene;
1 to 2% by weight of carnauba wax
1 to 5% by weight of VP/eicosene copolymer; and
1 to 5% by weight of triacontanyl PVP.

7. The composition of claim 2, wherein the combination of waxes comprises 1 to 5% by weight of beeswax.

8. The composition of claim 3, wherein the combination of film forming agents comprises 1 to 5% by weight of acrylates/ethylhexyl acrylate copolymer.

9. The composition of claim 4, wherein the combination of film forming agents comprises 1 to 5% by weight of polyvinylpyrrolidone.

10. The composition of claim 1, wherein the composition comprises:
0.02 to 2% by weight of silica;
0.01 to 1% by weight of tocopheryl acetate; and
0.01 to 1% by weight of panthenol.

11. The composition of claim 1, wherein the composition further comprises:
1 to 8% by weight of glyceryl stearate;
1 to 6% by weight of cellulose;
0.5 to 4% by weight of behenyl alcohol; and
0.5 to 3% by weight of propanediol.

12. The composition of claim 1, wherein the composition further comprises:
1 to 8% by weight of stearic acid;
1 to 8% by weight of hydrogenated cyclopentadiene; and
1 to 8% by weight of trisiloxane.

13. The composition of claim 1, wherein the composition further comprises:
0.01 to 1% by weight of potassium hydroxide;
0.01 to 1% by weight of phenoxyethanol;
0.01 to 1% by weight of polyimide-1;
0.01 to 1% by weight of disteardimonium hectorite;
0.01 to 1% by weight of caprylyl glycol;
0.01 to 1% by weight of hydroxyethylcellulose;
0.01 to 1% by weight of isopropyl titanium triisostearate; and
0.01 to 1% by weight of xanthan gum.

14. The composition of claim 1, wherein the composition further comprises:
0.01 to 1% by weight of laureth-21;
0.01 to 1% by weight of triethyl citrate;
0.01 to 1% by weight of disodium EDTA;
0.01 to 1% by weight of benzyl alcohol;
0.01 to 1% by weight of ethylhexylglycerin;
0.01 to 1% by weight of hexylene glycol;
0.01 to 1% by weight of aminomethyl propanol;
0.01 to 1% by weight of tocopherol; and
0.01 to 1% by weight of salicylic acid.

15. The composition of claim 1, wherein the composition further comprises:
45 to 55% by weight of isododecane;
3.5 to 5.5% by weight of quaternium-90 bentonite; and
0.75 to 1.25% by weight of propylene carbonate.

16. The composition of claim 1, wherein the composition further comprises:
5 to 10% by weight of polymethylsilsesquioxane;
1 to 5% by weight of rayon; and
0.01 to 1% by weight of phenoxyethanol.

17. The composition of claim 1, wherein the composition further comprises:
0.01 to 1% by weight of hydrogenated cyclopentadiene;
0.01 to 1% by weight of caprylyl glycol; and
0.01 to 1% by weight of isopropyl titanium triisostearate.

18. An apparatus for applying a mascara composition to an eyelash, the apparatus comprising a mascara wand and the mascara composition claim 1 in contact with the mascara wand.

19. A method of defining the appearance of eyelashes and increasing the length of eyelashes, the method comprising applying the mascara composition of claim 1 to the eyelashes, wherein application of the composition to the eyelashes defines the appearance of the eyelashes.

20. The method of claim 19, wherein the composition is applied to the eyelashes with a mascara wand.
